(19) United States
(12) Patent Application Publication
(10) Pub. No.: US 2024/0394604 A1
(43) Pub. Date: Nov. 28, 2024

(54) PERSONALIZED RIDE EXPERIENCE BASED ON REAL-TIME SIGNALS

(71) Applicant: Lyft, Inc., San Francisco, CA (US)

(72) Inventor: Gil Arditi, Palo Alto, CA (US)

(21) Appl. No.: 18/793,586

(22) Filed: Aug. 2, 2024

Related U.S. Application Data
(63) Continuation of application No. 15/851,078, filed on Dec. 21, 2017, now abandoned.

(51) Int. Cl. G06N 20/00; G16H 50/20

(52) U.S. Cl. CPC G06N 20/00; G16H 50/20

(57) ABSTRACT

In particular embodiments, a computing system may receive a ride request from a ride provider. The system may access a user preference profile of the ride requestor. The user preference profile may indicate at least one ride preference of the ride requestor. The system may match the ride requestor to a ride provider based on the at least one ride preference of the ride requestor. The system may send the at least one ride preference to a computing device of a vehicle associated with the ride provider. The system may determine a current location of each of the ride requestor and the ride provider. Responsive to determining that the current location is within a threshold distance, the system may instruct the computing device of the vehicle to adjust settings of one or more in-vehicle devices according to the at least one ride preference of the ride requestor.

### PRIORITY

This application is a continuation under 35 U.S.C. § 120 of U.S. patent application Ser. No. 15/851,078, filed 21 Dec. 2017, which is incorporated herein by reference.

### BACKGROUND

A transportation management system, in response to ride requests, may match the needs of ride requestors with ride providers who are willing to use their vehicles to provide the requested rides. For instance, through a transportation application installed on a mobile device, a ride requestor may request for a ride from a starting location to a destination at a particular time. In response to the request, the transportation management system may match the ride requestor's needs with any number of available ride providers and notify the matching ride providers of the ride request. The ride providers, through the transportation application installed on their respective mobile devices, may see the request notification and accept or reject the ride request. Once a ride provider accepts the request, the transportation management system may, in response, facilitate the ride transaction between the ride requestor and the ride provider.

Upon entering the vehicle of the dispatched ride provider, the ride requestor would usually experience a foreign environment. Not only would the vehicle cabin feel foreign, but so would various vehicle configurations related to passenger comfort and experience, such as the music being played, the cabin temperature, seat configurations, windows being opened or closed, etc. Since the dispatched vehicle's passenger configurations likely would not match the preferences of the ride requestor, the ride experience of the ride requestor is often less than ideal.

Another issue with conventional dispatched rides relates to safety. For example, a health-related emergency of the ride requestor may go unnoticed by the ride provider who is focused on driving.

### BRIEF DESCRIPTION OF THE DRAWINGS

FIG. 1 illustrates an example of a transportation management system for matching ride requestors with ride providers.

FIG. 2 illustrates an example configuration of devices in a provider's vehicle.

FIGS. 3A-3C illustrate an example of a transportation management vehicle device.

FIG. 4 illustrates an example of sensors positioned in a vehicle.

FIGS. 5A-5D illustrate an example of ride personalization according to particular embodiments.

FIG. 6 illustrates an example of a method for personalizing ride configurations based on known or predicted requestor preferences.

FIG. 7 illustrates an example block diagram for training a machine-learning model used for predicting requestor preferences.

FIG. 8 illustrates an example of a method for personalizing ride configurations based on real-time sensor data of the passenger compartment of a vehicle or other current contextual information.

FIG. 9 illustrates an example block diagram for training a machine-learning model used for predicting requestor preferences based on real-time sensor data of the passenger compartment of a vehicle or other current contextual information.

FIG. 10 illustrates an example of a method for detecting and responding to emergencies or other actionable events/incidents occurring within the passenger compartment of a vehicle.

FIG. 11 illustrates an example block diagram for training a machine-learning model used for predicting emergencies or other actionable events/incidents occurring within the passenger compartment of a vehicle.

FIG. 12 illustrates an example block diagram of a ride-service management environment.

FIG. 13 illustrates an example block diagram of a ride-service management environment for matching ride requestors with autonomous vehicles.

FIG. 14 illustrates an example of a computing system.

### DESCRIPTION OF EXAMPLE EMBODIMENTS

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described. In addition, the embodiments disclosed herein are only examples, and the scope of this disclosure is not limited to them. Particular embodiments may include all, some, or none of the components, elements, features, functions, operations, or steps of the embodiments disclosed above. Embodiments according to the invention are in particular disclosed in the attached claims directed to a method, a storage medium, a system and a computer program product, wherein any feature mentioned in one claim category, e.g., method, can be claimed in another claim category, e.g., system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

When a ride requestor enters a dispatched vehicle (human-driven or autonomous) of another, the vehicle is conventionally configured based on the preferences of its owner. Such pre-existing vehicle configurations may not be particularly suitable or desirable for the ride requestor and therefore may result in a suboptimal ride experience. For example, to the ride requestor, the music may be too loud or galling, the temperature may be too high, and the air may be stuffy due to the windows being closed. In order for the vehicle configurations to be adjusted to the liking of the ride requestor, the ride requestor would need to do so manually, whether by asking the human driver or interfacing with the vehicle directly. Even for ride requestors who know what they want to adjust, how to make the adjustment in a foreign vehicle, and are willing to go through the trouble, such an adjustment process would hamper the overall ride experience. The ride experience would be especially hampered in situations where, e.g., the number of desired adjustments is high and therefore tedious, the latency for a configuration adjustment to be appreciable is long (e.g., the interior temperature may take minutes to adjust to the desired level), the control interface in the vehicle is foreign, and/or language barriers exist between the ride requestor and the driver. Furthermore, certain configuration adjustments that could improve the ride experience for a requestor may not even cross the requestor's mind (e.g., the ride requestor may be occupied or unaware of certain configuration options), and as a result, the adjustments would not be acted upon.

Another shortcoming of conventional dispatched rides relates to passenger and driver safety. For example, health emergencies, conflict between ride requestors and/or providers, and/or behavior that is against policy (e.g., vandalism, inappropriate activity, etc.) may go unnoticed as the driver may be focused on driving or the vehicle may be so large that it is difficult to monitor the behavior of the one or more requestors. Additionally, as autonomous vehicles become more ubiquitous, a human driver may not be present in a vehicle in the case of an emergency.

Particular embodiments described herein relate to systems, apparatuses, and methods for providing personalized ride experiences to users of a transportation management system. A transportation management system may facilitate ride sharing between ride providers and ride requestors and/or manage a fleet of autonomous vehicles to fulfill transportation needs of ride requestors. To improve the ride experience for ride requestors, the transportation management system may leverage the system's knowledge of ride requestors to determine ride preferences, which may be personalized for individual ride requestors, and instruct devices associated with the ride provider to actuate those ride preferences, such as playing the preferred type of music and/or adjusting the temperature to a preferred level. In particular embodiments, sensors in the vehicle may be used to capture real-time sensor data associated with the ride requestor. Real-time sensor data may include, for example, video, image, audio, infrared, temperature, 3D modeling, and any other suitable types of data that capture the ride requestor's current state. In particular embodiments, the real-time sensor data may be processed using one or more machine-learning models trained based on similar types of data to predict real-time features of the ride requestor. The real-time features may include, for example, the ride requestor's current mood (e.g., happy, angry, sad, etc.), stress level, comfort level with respect to vehicle amenities (e.g., temperature, audio, entertainment, etc.), health condition, and/or any other features that may characterize or represent the requestor's current state.

Current information about the particular ride requestor may be used to improve the overall ride experience for the ride requestor and/or provide additional services. For example, currently detected sensor data and/or previously known information about the ride requestor may be used to predict the ride requestor's likes and dislikes, which in turn may be used to configure the vehicle (e.g., entertainment, air-conditioning setting, lighting, windows, etc.). As another example, information about the ride requestor and the ride provider may be used to trigger translation services to help bridge any language barriers. Further, access to a ride requestor's calendar and/or task-tracking system may enable the system to provide the ride requestor with productivity-related services, such as reminding the ride requestor of upcoming events/tasks. As yet another example, interior sensor data may be used to detect and respond to emergencies appropriately (e.g., urgent health conditions, etc.). The various embodiments described herein, therefore, enable vehicles dispatched for transporting ride requestors to be dynamically configured to suit the preferences of the ride requestors and provide additional services such as emergency detection. Such enhancements enable the dispatched vehicles to provide ride requestors with improved ride experience and safety.

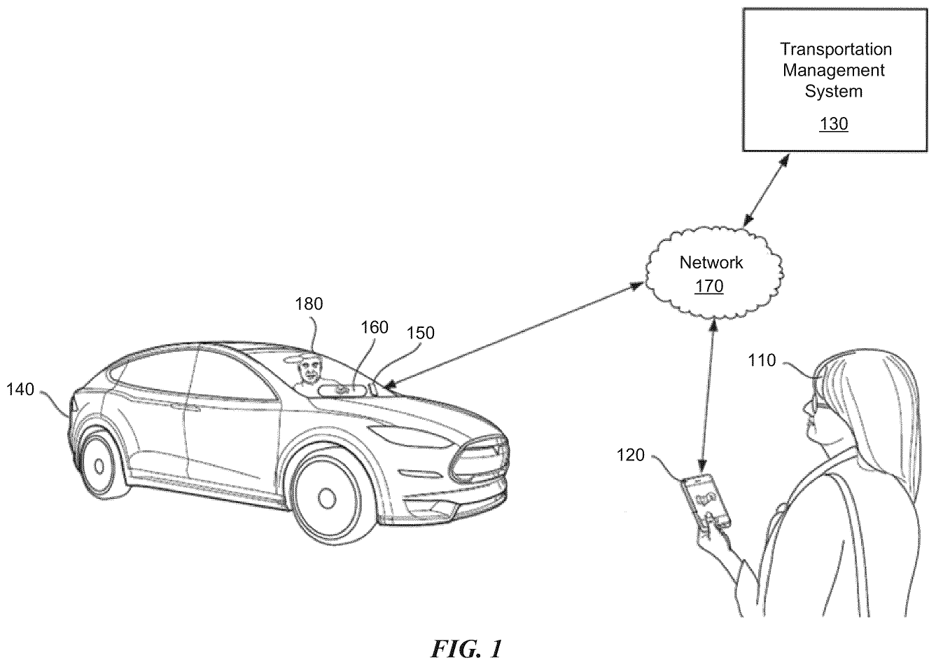

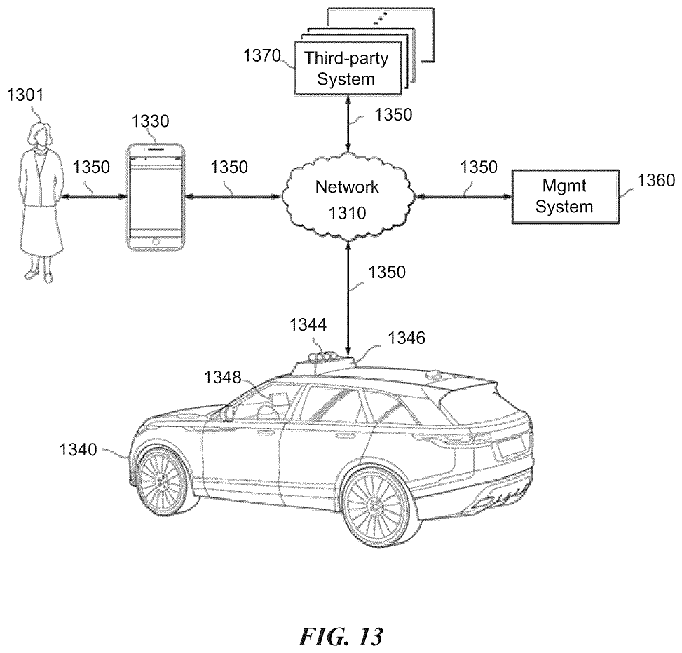

FIG. 1 illustrates an example of a transportation management system 130 for matching ride requestors 110 and ride providers 180, in accordance with particular embodiments described herein. The transportation management system 130 may be configured to communicate with both the requestor's 110 computing device 120 and the provider's 180 computing device 150. The provider computing device 150 may be configured to communicate with a transportation management vehicle device 160 that is configured to easily and efficiently provide information to a provider 180 and/or a requestor 110, obtain internal sensor data pertaining to the passenger compartment of the vehicle 140, and/or adjust configurations of the vehicle 140. While FIG. 1 illustrates an example where the dispatched vehicle 140 is a conventional human-driven vehicle, embodiments described herein may also be applied to autonomous vehicles, which would be described in further detail below, such as with reference to FIG. 13.

In particular embodiments, the requestor 110 illustrated in FIG. 1 may use a transportation application running on a requestor computing device 120 to request a ride from a specified pick-up location to a specified drop-off location. The request may be sent over a communication network 170 to the transportation management system 130. The ride request may include request information, which may include, for example, an identifier associated with the requestor and/or the requestor computing device, user information associated with the requestor, a location of the requestor computing device at the time of the request, a requested time for the ride (e.g., at a scheduled future time or an instant/current time), and/or any other relevant information for matching the ride request with ride providers as described herein. The ride request may also include transport information, such as, e.g., a pick-up location, a drop-off location, a “best fit/predictive” location (e.g., a particular location in the origination/destination region suitable for pick-up/drop-off at a given time), preferred pick-up/drop-off location type (e.g., a curb segment), or any other suitable information for indicating the requestor's transportation preferences and/or objectives. In particular embodiments, the ride request may further include any other preferences or needs of the requestor, including, for example, navigation preferences (e.g., highways vs. local streets; particular routes; stop overs), music or entertainment preferences (e.g., link to a music playlist or station hosted by a 3rd-party music provider, news station, etc.), personalized pattern/color to display on a transportation management vehicle device to help the ride provider and requestor identify each other, particular vehicle features or restrictions (e.g., pet friendly, child seat, wheelchair accessible, maximum/minimum passenger or cargo compartment, etc.).

In particular embodiments, the transportation management system 130 may, in response to a ride request, identify available providers that are registered with the transportation management system 130 through an application installed on each of their respective mobile computing devices 150 or through an associated transportation management vehicle device 160. For example, the transportation management system 130 may locate candidate ride providers 180 who are available (e.g., based on a status indicator provided through each ride provider's 180 computing device 150) and in the general vicinity of the requested pick-up location (e.g., based on GPS data provided by the provider computing device 150 and the requestor computing device 120). The system 130 may further access various information about each candidate ride provider 180, including, for example, vehicle features (e.g., vehicle type, size, class, etc.), amenities, limitations of the vehicle, route for transporting other passengers in the vehicle in a ride-sharing scenario (e.g., the ride provider 180 may be in the process of transporting different, unrelated ride questors), schedule information regarding the ride provider's 180 future availability, diagnostics associated with the vehicle (e.g., gas level, battery level, engine status, etc.), and/or any other suitable information. In particular embodiments, the transportation management system 130 may match the information pertaining to each candidate ride provider 180 with the preferences/requirements specified in the ride request (e.g., preferred vehicle type/size, pick-up and drop-off locations, travel time constraints, etc.) and assign the candidate ride provider 180 a score that represents how good the match is. In particular embodiments, the transportation management system 130 may rank the candidate ride providers 180 based on their respective scores. In particular embodiments, the transportation management system 130 may select a number (e.g., 3, 5, 10, etc.) of top-ranking candidate ride providers 180 and inquire whether any of them is willing to fulfill the ride request. In particular embodiments, the system 130 may send notifications to those ride providers 180 one by one, in the order of their rankings (e.g., starting with the highest-ranked or best-matched one), until someone accepts. Alternatively, the system 130 may simultaneously send notifications to the selected top-ranking ride providers 180 and assign the first ride provider 180 who accepts to fulfill the ride request.

In particular embodiments, different types of information may be sent to the ride provider 180 and the ride requestor 110 during different stages of the ride-matching process. For example, the aforementioned notification for inquiring whether a ride provider 180 is interested in fulfilling a ride request may include, for example, the pick-up location of the ride request, estimated time of travel, fees for the ride, particular ride requirements (e.g., car seat availability), the ride requestor's 110 rating on the system 130, and any other pertinent information that would allow the ride provider 180 to make an informed decision as to whether to accept or reject the ride request. Upon seeing the notification, the provider 180 may accept or reject the ride request through the provider communication device 150. In particular embodiments, the provider computing device 150 may notify the transportation management system 130 that the provider 180 received the notification and further inform the system 130 of whether the provider 180 accepted or rejected the request. The information sent to the system 130 may include, for example, an acceptance indicator (e.g., a flag) and current location of the ride provider 180. In particular embodiments, the provider 180 may not be required to explicitly accept the request. For instance, the provider 180 may enter a mode where the provider 180 agrees to accept all requests that are sent to the provider 180 without the ability to decline and/or review requests before accepting. Once a ride provider 180 accepts the ride request, the transportation management system may send the ride provider 180 additional information, such as the requestor's 110 profile information (e.g., name, profile picture, etc.), destination information, route from the requested origination location to the destination locations, navigation instructions to the pick-up location, and any other suitable information that would help the ride provider 180 fulfill the ride request.

In particular embodiments, after a ride provider 180 accepted the ride request, the transportation management system 130 may provide the ride requestor 110 information pertaining to the ride provider 180. The information may include, for example, the ride provider's 180 profile information (e.g., name, representative symbol or graphic, social-media profile picture, rating, past ride history and reviews, etc.), a suggested route from the requested origination location to the destination location, tracking information that indicates the ride provider's 180 current location, estimated fare, and/or any other relevant information that facilitates the transaction and informs the ride requestor 110 of what to expect.

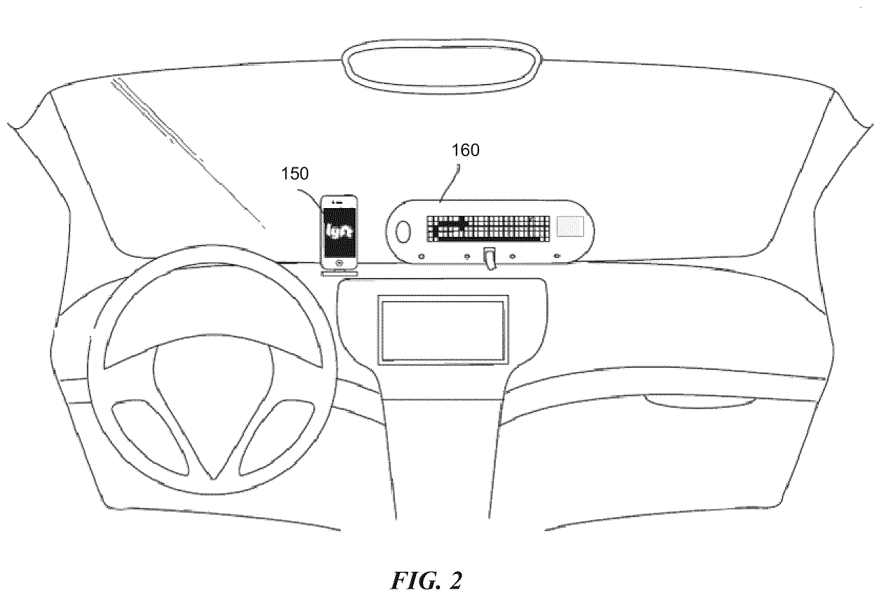

FIG. 2 illustrates an example configuration of devices in a provider's vehicle. In the example shown, the provider computing device 150 is held by a mobile device holder so that the display of the provider computing device 150 is visible to the ride provider (or driver) and any of the other passengers in the vehicle. A transportation management vehicle device 160 may be similarly positioned so that its interior interface can be seen by the passengers. In particular embodiments, the provider computing device 150 and/or the transportation management vehicle device 160 may both include sensors for monitoring the passenger compartment of the vehicle (as used herein, “passenger compartment” refers to the area of a vehicle designed and intended for the seating of the driver and other passengers). Examples of sensors may include cameras for capturing visible data; microphones for capturing audible data; infrared sensors for detecting heat emitted by passengers; gyroscopes and accelerometers for detecting vehicle motion; and any other sensors suitable for detecting environmental signals occurring in the passenger compartment of the vehicle. In particular embodiments, such sensors may be integrated with the vehicle (e.g., traditional human-driven vehicles or autonomous vehicles) or with a detachable device, such as the transportation management vehicle device 260. The sensors may be located at any suitable location, such as in the upper corners of the passenger compartment, the dashboard, seats, side doors, ceiling, rear view mirror, central console, floor, or any other location where the sensor would be effective in detecting the type of signals it is designed for.

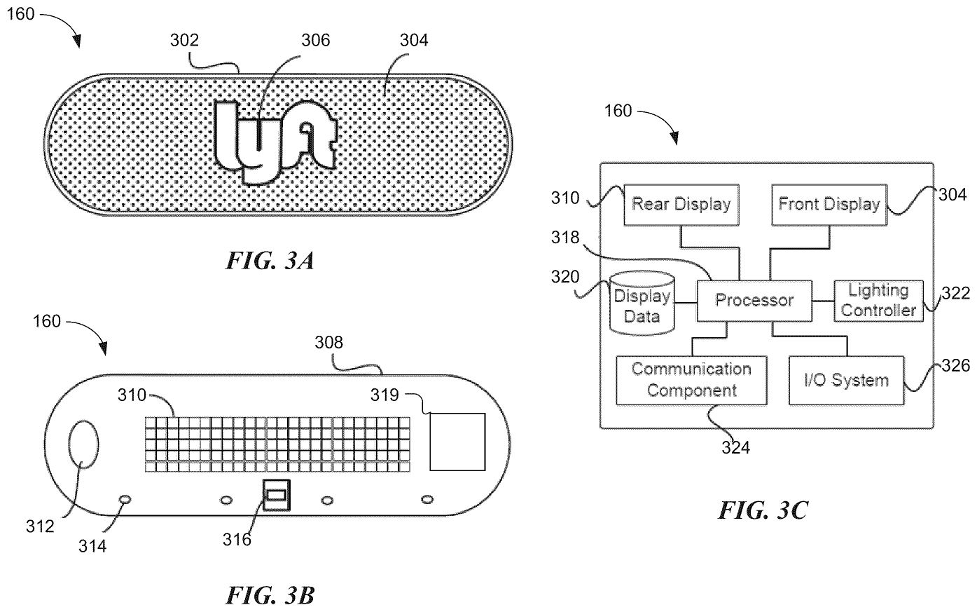

FIGS. 3A-3C show an example transportation management vehicle device 160 in accordance with embodiments described herein. The transportation management vehicle device 160 may include a front view 302 (FIG. 3A) and a rear view 308 (FIG. 3B). In particular embodiments, the front view 302 may be designed to face the outside of the vehicle so that it is visible to, e.g., ride requestors, and the rear view 308 may be designed to face the interior of the vehicle so that it is visible to, e.g., the passengers. As shown in FIG. 3A, a front view 302 of the transportation management vehicle device 160 may include a front display 304. In particular embodiments, the front display 304 may include a secondary region or separate display 306. As shown in FIG. 3A, the front display 304 may include various display technologies including, but not limited to, one or more liquid crystal displays (LCDs), one or more arrays of light emitting diodes (LEDs), AMOLED, or other display technologies. In particular embodiments, the front display 304 may include a cover that divides the display into multiple regions. In particular embodiments, separate displays may be associated with each region. In particular embodiments, the front display 304 may be configured to show colors, text, animation, patterns, color patterns, or other identifying information to requestors and other users external to a provider vehicle (e.g., at a popular pick-up location, requestors may quickly identify their respective rides and disregard the rest based on the identifying information shown). In particular embodiments, the secondary region or separate display 306 may be configured to display the same, or contrasting, information as front display 304.

FIG. 3B shows an embodiment of the rear view 308 of the transportation management vehicle device 160. As shown, the rear view 308 in particular embodiments may include a rear display 310. As with the front display 304, the rear display 310 may include various display technologies including, but not limited to, one or more liquid crystal displays (LCDs), one or more arrays of light emitting diodes (LEDs), AMOLED, or other display technologies. The rear display 380 may be configured to display information to the provider, the requestor, or other passengers in the passenger compartment of the vehicle. In particular embodiments, rear display 310 may be configured to provide information to people who are external to and behind the provider vehicle. Information may be conveyed via, e.g., scrolling text, color, patterns, animation, and any other visual display. As further shown in FIG. 3B, the transportation management vehicle device 160 may include a power button 312 or any other user interface which can be used to turn the device 160 on or off. In particular embodiments, power button 312 may be a hardware button or switch that physically controls whether power is provided to the transportation management vehicle device 160. Alternatively, power button 312 may be a soft button that initiates a startup/shutdown procedure managed by software and/or firmware instructions. In particular embodiments, the transportation management vehicle device 160 may not include a power button 312. Additionally, the transportation management vehicle device 160 may include one or more light features 314 (such as one or more LEDs or other light sources) configured to illuminate areas adjacent to the device 160 and/or provide status signals.

In particular embodiments, the transportation management vehicle device 160 may include a connector 316. In particular embodiments, the connector 316 may be configured to physically connect to the ride provider's computing device and/or the requestor's computing device. In particular embodiments, the connector 316 may be configured for physically connecting the transportation management vehicle device 160 to the vehicle for power and/or for communicating with the vehicle. For instance, the connector 316 may implement the CANvas interface or any other suitable communication interface or protocol for communicating with a vehicle. In another instance, the connector 316 may include a controller area network (CAN) bus interface that may be utilized in communicating with a vehicle. For example, the CAN bus interface may interface with an on-board diagnostics (OBD) port (e.g., an OBD-I port, an OBD-II port, etc.) of the vehicle. In particular embodiments, through the connector 316, the transportation management vehicle device 160 may be able to issue instructions to the vehicle's onboard computer and cause it to adjust certain vehicle configurations, such as air-conditioning level, entertainment/informational content (e.g., music, news station, content source, etc.), audio volume, window configuration, seat warmer temperature, and any other configurable features of the vehicle. As another example, the connector 316 may enable the transportation management vehicle device 160 to query the vehicle for certain data, such as current configurations of any of the aforementioned features, as well as the vehicle's speed, fuel level, tire pressure, external temperature gauge, navigation system, and any other information available through the vehicle's computing system. In particular embodiments, the transportation management vehicle device 160 may be further configured with wireless communication capabilities (e.g., Bluetooth, WI-FI, NFC, etc.), thereby enabling the device 160 to wirelessly communicate with the vehicle, the provider's computing device, and/or the requestor's computing device.

In particular embodiments, the transportation management vehicle device 160 may be integrated with one or more sensors 319, such as a camera, microphone, infrared sensor, gyroscope, accelerometer, and any other suitable sensor for detecting signals of interest within the passenger compartment of the vehicle. For example, the sensor 319 may be a rear-facing wide-angle camera that captures the passenger compartment and any passengers therein. As another example, the sensor 319 may be a microphone that captures conversation and/or sounds in the passenger compartment. The sensor 319 may also be an infrared sensor capable of detecting motion and/or temperature of the passengers.

Although FIG. 3B illustrates a particular number of components (e.g., a single sensor 319, a single display 310, a single connector 316, etc.), one of ordinary skill in the art would appreciate that any suitable number of each type of component may be included in the transportation management vehicle device 160. For example, in particular embodiments, a transportation management vehicle device 160 may include one or more of a camera, microphone, and infrared sensor. As another example, the device 160 may include one or more communication interfaces, whether wired or wireless.

FIG. 3C shows a block diagram of various components of a transportation management vehicle device 160 in accordance with particular embodiments. As shown in FIG. 3C, the transportation management vehicle device 160 may include a processor 318. Processor 318 may control information displayed on rear display 310 and front display 304. As described herein, each display may be designed to display information to different intended users, depending on the positioning of the users and the transportation management vehicle device 160. In particular embodiments, display data 320 may include stored display patterns, sequences, colors, text, animation or other data to be displayed on the front and/or rear display. The display data 320 may also include algorithms for generating content and controlling how it is displayed. The generated content, for example, may be personalized based on information received from the transportation management system, any third-party system, the vehicle, and the computing devices of the provider and/or requestor. In particular embodiments, display data 320 may be stored on a hard disk drive, solid state drive, memory, or any other storage device.

In particular embodiments, lighting controller 322 may manage the colors and/or other lighting displayed by light features 314, the front display 304, and/or the back display 310. The lighting controller may include rules and algorithms for controlling the lighting features 314 so that the intended information is conveyed. For example, to help a set of matching provider and requestor find each other at a pick-up location, the lighting controller 322 may obtain instructions that the color blue is to be used for identification. In response, the front display 304 may display blue and the lighting controller 322 may cause the light features 314 to display blue so that the ride provider would know what color to look for.

In particular embodiments, the transportation management vehicle device 160 may include a communication component 324 for managing communications with other systems, including, e.g., the provider device, the requestor device, the vehicle, the transportation management system, and third-party systems (e.g., music, entertainment, traffic, and/or maps providers). In particular embodiments, communication component 324 may be configured to communicate over WI-FI, Bluetooth, NFC, RF, or any other wired or wireless communication network or protocol.

In particular embodiments, ride-service device 160 may include an input/output system 326 configured to receive inputs from users and/or the environment and provide output. For example, I/O system 326 may include a sensor such as an image-capturing device configured to recognize motion or gesture-based inputs from passengers, a microphone configured to detect and record speech or dialog uttered, a heat sensor to detect the temperature in the passenger compartment, and any other suitable sensor. The I/O system 326 may output the detected sensor data to any other system, including the transportation management system, the computing devices of the ride provider and requestor, etc. Additionally, I/O system 326 may include an audio device configured to provide audio outputs (such as alerts, instructions, or other information) to users and/or receive audio inputs, such as audio commands, which may be interpreted by a voice recognition system or any other command interface. In particular embodiments, I/O system 326 may include one or more input or output ports, such as USB (universal serial bus) ports, lightning connector ports, or other ports enabling users to directly connect their devices to the transportation management vehicle device 160 (e.g., to exchange data, verify identity information, provide power, etc.).

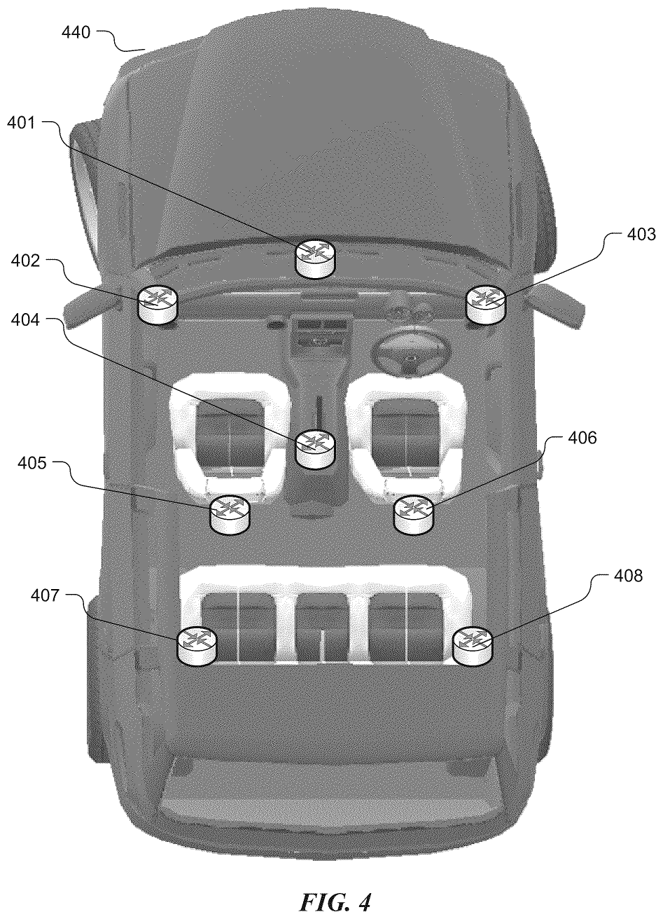

FIG. 4 illustrates an example of sensors 401-408 positioned in a vehicle 440, which may be a conventional human-driven vehicle or an autonomous vehicle. The sensors 401-408 may be any combination of any sensor types, including, for example, cameras, LiDARs, radio transceivers, infrared sensors, ultrasound sensors, temperature sensors, microphones, light detectors, motion detectors (including accelerometers, gyroscopes, etc.), weight sensors, odor or scent detectors, and any other suitable sensor types for monitoring the passenger compartment of the vehicle 440. A sensor may be located anywhere in the vehicle and could be integrated into the body of the vehicle 440 and/or attached externally to a surface of the vehicle 440. The particular placement of a sensor may depend on the type of sensor data that the sensor is designed to measure. As an example and not by way of limitation, FIG. 4 shows eight sensors 401-408 positioned in various locations in the vehicle 440. For example, sensor 401 may be a camera positioned on the dashboard of the vehicle 440 to capture full sensory data of passengers sitting in the front of the vehicle and also partial sensory data of passengers sitting in the back. Sensors 402 and 403, which are positioned in the front corners of the vehicle 440, may be configured to specifically capture sensor data, such as images and audio, associated with the front-left passenger and the front-right passenger, respectively. Similarly, sensors 405 and 406, which are positioned behind the front-left seat and the front-right seat, respectively, may be configured to capture sensor data, such as images and audio, associated with the passengers sitting in the back of the vehicle 440. Sensor 404, which may be configured to measure the environment or surroundings in the passenger compartment (e.g., ultrasound, LiDAR, temperature sensor, odor sensor, etc.), may be positioned over the center console of the vehicle 440. Sensor 407 and 408, which are positioned in the back-left and back-right corners of the vehicle 440, may be configured to detect passenger entry into the vehicle (e.g., based on visual images, magnetic entry sensors, weight sensors in the seats, etc.).

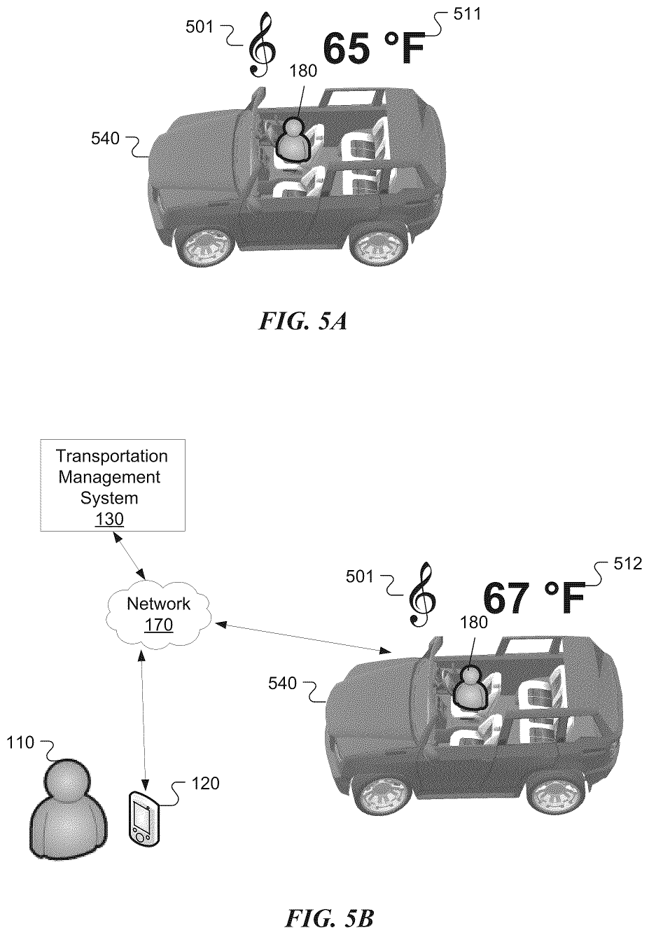

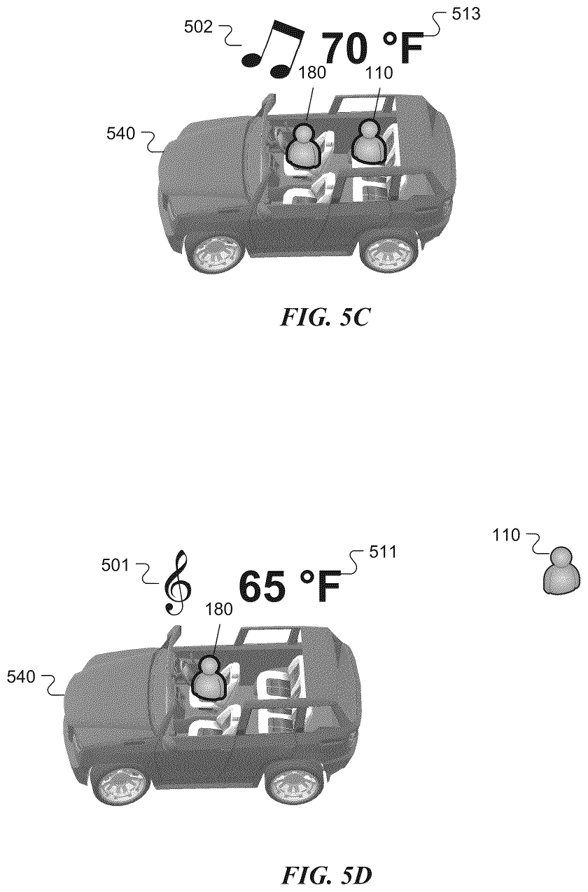

FIGS. 5A-5D illustrate an example of ride personalization according to particular embodiments. FIG. 5A illustrates an example of a ride provider 180 driving a vehicle 540 (alternatively, the vehicle could be an autonomous vehicle without a human driver) prior to being dispatched to service a ride request. At this time, the vehicle 540 may have certain pre-existing configurations, such as music 501 and interior temperature 511. For example, the ride provider 180 may be listening to heavy-metal music 501 and set the interior temperature 511 to be 65° F. If the vehicle 540 is instead an autonomous vehicle, the pre-existing configurations for music 501 and temperature 511 may simply be off.

FIG. 5B illustrates an example where the vehicle 540 has been matched by the transportation management system 130 with a ride requestor 110. In the scenario shown, the vehicle 540 is en route to pick up the ride requestor 110. In particular embodiments, while the vehicle 540 is en route, personal information (e.g., music and temperature preferences) associated with the ride requestor 110 may be transmitted to a computing device (not shown) of the vehicle 540. The computing device may be an in-vehicle computing device that is integrated with the vehicle or any type of removable/detachable computing device, such as a mobile device or transportation management vehicle device 160. The personal information may be sent from the transportation management system 130 and/or the computing device 120 of the ride requestor 110 to the vehicle 540 through a network 170. In particular embodiments, the vehicle 540 may selectively adjust its configurations based on the received personal information of the ride requestor 110. For example, since music settings may be changed instantaneously, the existing music 501 may remain unchanged (e.g., heavy-metal) until the ride requestor 110 is closer or within a threshold proximity (e.g., in terms of travel distance and/or travel time) from the vehicle 540. For instance, the existing music 501 may not change until the vehicle 540 and the ride requestor 110 are within 0.1 miles or 1 minute of each other, or until the ride requestor 110 enters the vehicle 540 or opens the door. Unlike music, the internal temperature takes time to adjust. As such, in particular embodiments the internal temperature may begin to adjust earlier, such as immediately when the vehicle 540 receives the personal information of the ride requestor 110 or when the vehicle 540 is within a threshold proximity from the ride requestor 110 (e.g., within 2 miles or 5 minutes). In particular embodiments, the particular threshold proximity may be determined based on the typical amount of time needed for temperature to adjust from the existing value to the desired value (e.g., the transportation management system 130 may determine the suitable threshold proximity based on observed historical times needed for various temperature adjustments). For example, if the difference between the temperature preferred by the ride requestor 110 and the existing temperature is 5° F., the threshold proximity that triggers a temperature change may be 8 minutes, whereas if the temperature difference is only 2° F., the triggering threshold proximity may be less, such as 3 minutes. In the scenario shown in FIGS. 5A-5D, the ride requestor 110 prefers an internal temperature of 70° F. As such, even though the vehicle 540 is still en route to pick up the ride requestor 110, the temperature has already begun to change. At the instant captured in FIG. 5B, the vehicle's 540 internal temperature 512 is at 67° F., rising from 65° F. to the desired 70° F.

FIG. 5C illustrates an example where the vehicle 540 has picked up the ride requestor 110 and is driving to the requested destination. In particular embodiments, shortly before the ride requestor 110 is picked up or when the ride requestor 110 enters the vehicle 540, the vehicle 540 changed the music setting to the type of music 502 preferred by the ride requestor 110 (e.g., classical music). In addition, by the time the ride requestor 110 is picked up, the internal temperature has reached the preferred temperature 513 of 70° F. As shown, rather than having the ride requestor 110 enter an environment where the temperature is much colder and the music is much louder than preferred, the ride requestor 110 enters an environment where both the temperature and music are as preferred.

In particular embodiments, once the ride requestor 110 has been picked up, real-time sensor data about the ride requestor 110 may be gathered and used to further personalize the ride experience for the ride requestor 110. Real-time sensor data may be captured by any suitable sensor types within the vehicle, such as those described with reference to FIG. 4. For example, cameras may capture images or videos of the ride requestor 110, microphones may detect speech, and temperature sensors may detect the body temperature of the ride requestor 110. The real-time sensor data may more accurately reflect the current state and preferences of the ride requestor 110 than the pre-stored personal information. Thus, in particular embodiments the computing device of the vehicle 540 may process the real-time sensor data to predict the ride requestor's 110 current preferences.

Continuing with the example provided above, even if the internal vehicle temperature is adjusted to 70° F. (i.e., the preferred temperature stated in the ride requestor's 110 personal information), real-time sensor data may reveal that the ride requestor 110 is in fact uncomfortably hot or cold. For example, from the real-time sensor data, the vehicle's computing device may extract various features that are associated with a person being hot, such as body temperature that is higher than a threshold, perspiration, certain body language/gestures (e.g., self-fanning, panting, etc.), and warm clothing. Similarly, the vehicle's computing device may also extract various features that signify that a person is cold, such as body temperature that is lower than a threshold, shivering, goosebumps, certain body language/gestures (e.g., self-hugging, rubbing hands together, breathing into hands, etc.), and light clothing. Based on such information extracted from the real-time sensor data, the vehicle 540 may further adjust its internal temperature accordingly. As another example, even if the currently playing music is set to the ride requestor's 110 preference as indicated in the requestor's 110 personal information, the requestor 110 may in fact not be enjoying the music. The ride requestor's 110 negative reactions may be signified by real-time sensor data capturing the ride requestor 110 putting on headphones or cringing when a song begins. In response, the vehicle 540 may turn off the music, lower the volume, or switch to a different genre. If instead the vehicle 540 detects real-time positive reactions from the ride requestor 110 (e.g., the ride requestor bobbing to the music), then the vehicle 540 may continue to play the music, play similar music, and/or raise the volume. Real-time sensor data may also reveal that the ride requestor 110 may currently be stressed (e.g., nail-biting, frowning, sighing, etc.) or tired (e.g., closed eyes, head resting on the head rest, etc.). While such information may not directly indicate whether the ride requestor likes or dislikes the music, the information may nevertheless be used to as evidence or corroborating evidence that music is likely unwelcomed and should be turned off.

FIG. 5D illustrates an example where the vehicle 540 has dropped off the ride requestor 110. In particular embodiments, the transportation management system 130 may determine that the ride requestor 110 is no longer in the vehicle 540 via their respective GPS locations, or the vehicle 540 may detect that the ride requestor 110 has exited based on, e.g., an entry sensor detecting the door opening, a weight sensor detecting that a previously occupied seat is no longer occupied, and/or computer vision determining that the ride requestor 110 is no longer present in the passenger compartment of the vehicle 540. Upon determining that the ride requestor 110 has exited the vehicle 540, the vehicle configurations that were changed to accommodate the ride requestor 110 may revert to their prior state. For example, the vehicle 540 may resume playing the music 501 that was playing before the vehicle 540 was dispatched to pick up the ride requestor 110. The vehicle 540 may also set the temperature 511 to be what it was before (e.g., 65° F.).

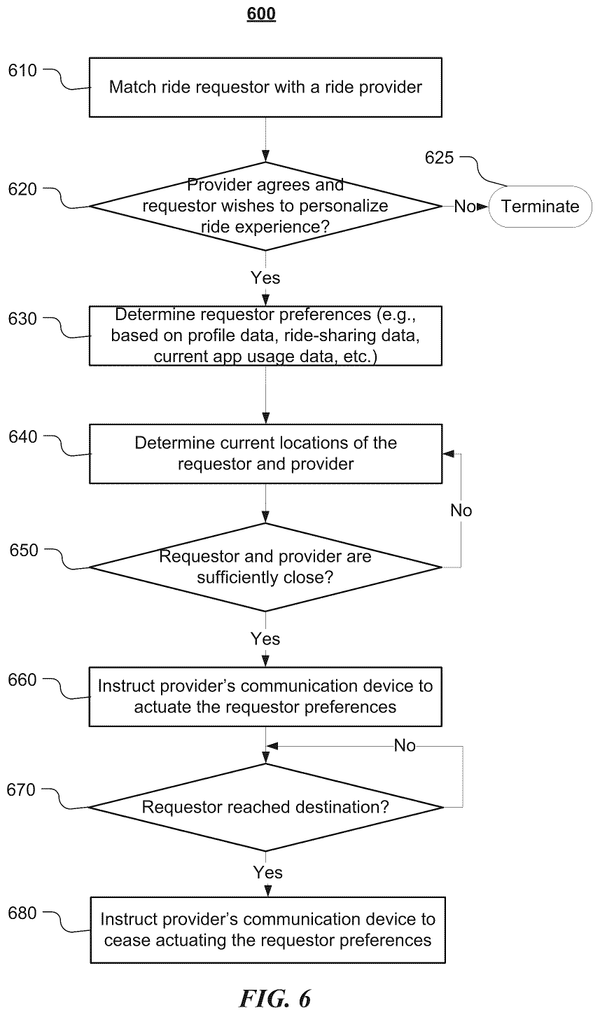

FIG. 6 illustrates a method 600 for personalizing ride configurations based on known or predicted requestor preferences, in accordance with particular embodiments. As an example, upon entering a vehicle (which may be a traditional human-driven vehicle or an autonomous vehicle), ride requestors may be greeted in their preferred language, their preferred audio station or playlist may be playing at the appropriate volume, and the temperature in the passenger compartment of the vehicle may be set to their preferences. The automated process for personalizing these and other ride configurations may begin at step 610, at which the transportation management system may match a ride requestor with a ride provider, according to particular embodiments described herein. In particular embodiments, the transportation management system may first match a ride requestor with a ride provider (e.g., based on proximity, availability, vehicle type, travel destination, etc.), and then proceed with personalizing the ride experience as illustrated in FIG. 6. Once a match is made, the transportation management system would know the identities of the ride requestor and the ride provider and may proceed with personalization operations.

While FIG. 6 illustrates an embodiment where matching is performed prior to assessing personalization factors between the matched ride requestor and provider, in other embodiments the transportation management system may take into consideration the personalization factors from the outset when matching the ride requestor with providers. For example, the ride requestors may indicate that they prefer ride providers who offer personalized rides and may specify the types of configurations that they would like to personalize (e.g., music, temperature, etc.). These preferences of the requestor may be persisted by the transportation management system (or the associated transportation application), along with any other profile information, or the preferences may be specified at the time the ride requestor is requesting the ride via the transportation application. During the matching process, the transportation management system may take into consideration whether a candidate ride provider offers the types of ride personalization desired by the ride requestor. For example, the matching algorithm may rank a candidate ride provider higher if the ride provider offers the desired ride personalization configurations associated with the request or requestor personalization settings/history.

At step 620, the system in particular embodiments may determine whether the ride provider agrees to provide a personalized ride experience and whether the ride requestor wishes for a personalized ride experience. In particular embodiments, the ride providers may have expressed that they are willing (or unwilling) to offer personalized ride experiences to their passengers obtained through the transportation management system. In addition, the ride providers may specify the particular types of personalization that they are willing (or unwilling) to accommodate. For example, the providers may indicate that they are willing to offer any subset (including a null set or the whole set) of the following personalized ride configurations: audio content (e.g., music genre, station, or playlist, audiobook, news, sports, talk radio, weather, and any other content), video content (e.g., shows, channels, movies, news, sports, or any other content), games, preferred temperature, preferred language, seat configuration (e.g., front or back seat, amount of legroom, degree of incline/decline, seat warmer temperature, etc.), amount in which the windows or moon roof should be opened (e.g., fully closed, slightly open, fully open, etc.), whether freeways/highways should be avoided, maximum speed limit, and any other vehicle configurations that may be automatically controlled. In particular embodiments, the transportation management system may also determine whether the ride requestor desires personalized rides, or the system may assume that personalized rides are preferred (or not preferred) if no preference is otherwise specified. In particular embodiments, previously expressed preferences of the ride provider and ride requestor may have been persisted by the transportation management system or the transportation application installed on their respective devices. The ride provider and ride requestor may also have specified their respective preference when making or responding to the ride request (e.g., via the user interface of the transportation application). In particular embodiments, if either (1) the ride provider does not offer personalized rides or the types of personalized configurations requested by the ride requestor, or (2) the ride requestor does not wish for a personalized ride, the process may terminate 625. On the other hand, if the ride provider offers personalized rides and the ride requestor so desires, then the process may continue on to step 630.

At step 630, the transportation management system may determine how to personalize the various ride configurations that the ride requestor desires and the ride provider offers. For example, once the system determines that the vehicle's audio output may be personalized, the system may determine what music or news, if any, the requestor would like to listen to during the ride, and/or the preferred audio volume level. In particular embodiments, certain user preferences may be explicitly specified by the ride requestor. For example, the ride requestors may have saved their preferences with the transportation management system or the associated transportation application. As another example, the specific preferences may be saved on a third-party system accessible by the transportation management system. For instance, the ride requestors may provide the transportation management system their credentials for accessing a third-party system that stores the ride requestor's music collection, playlist, or station. Using the credentials, the transportation management system may access the personalized information stored on the third-party system and use the information to actuate the personalized vehicle configurations.

In particular embodiments, machine learning may be used to extract actionable information from observed signals as well as the requestor's profile information. Any suitable machine-learning models may be used, including neural networks, decision trees, clustering algorithms, and any other suitable machine-learning techniques. In particular embodiments, the machine-learning models may be trained on historical user data. The training data may include a sufficiently large number (e.g., hundreds, thousands, etc.) of training data samples, which may include a variety of features that may correlate to user preferences, whether or not such correlation is known. Any data relating to the ride requestor may be processed by the machine-learning model, and the machine-learning model through training may determine what information is relevant and what information is not. The training data may, for example, be labeled with known personalization preferences. For example, the label of one training data point may indicate that the requestor prefers classical music, warm temperature, and no conversation with the driver. The label of another training data point may indicate that another requestor (or the same requestor) prefers pop music and welcomes conversation. These training data points may include a variety of information associated with their respective requestors, such as those described in further detail below. Based on the training data points, a machine-learning model may automatically learn which features within the provided data were predictive of the preferred personalization and weigh them accordingly.

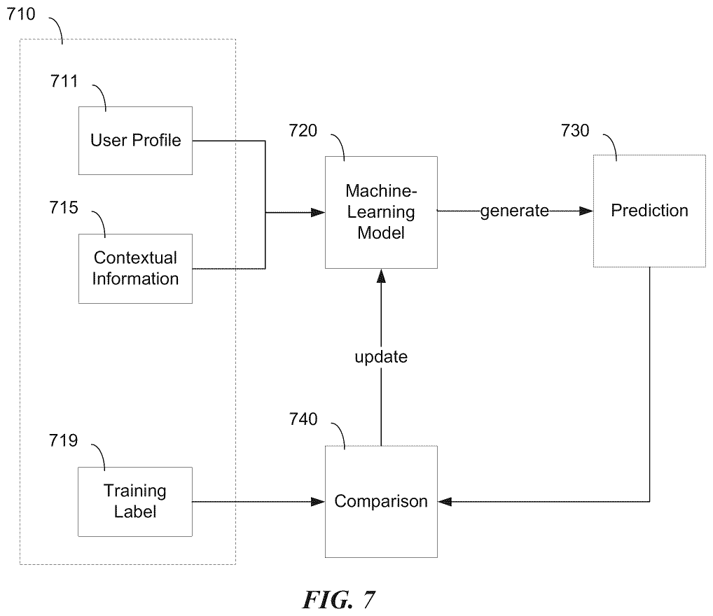

FIG. 7 illustrates an example block diagram for training a machine-learning model 720. The machine-learning model 720 may take as input any data associated with the ride requestors and learn to identify features within the data that are predictive of personalization preferences. FIG. 7 illustrates an example of one training data sample 710 in a set of training data that may be used to train the machine-learning model 720. The training data sample 710 may include, for example, the ride requestor's profile information 711, such as the ride requestor's age, gender, residence location, work location, ride-sharing patterns (e.g., frequently visited locations, average commute path, average commute times, average commute length, common pickup/drop-off locations and times, the requestor's location x minutes before being picked up and y minutes after being picked up, etc.), reviews and ratings submitted through the transportation management system, vehicle and feature preferences (e.g., luxury or standard vehicles, large or small vehicles, passenger capacity, child car seats, etc.), express preferences for personalizing ride configurations (e.g., preferred music genre, preferred news station, temperature, window configuration, etc.), and any other suitable data relating to the ride requestor.

In particular embodiments, the training data sample 710 may also include current contextual information 715 relating to the ride request. This may include, for example, the current weather condition or temperature, the time of day, traffic condition in the region, etc. The system may also garner contextual information 715 from the requestor's device. For example, through the transportation application installed on the device, the transportation management system may know how the application was used when the ride request was made. For example, the requestor's pattern of tapping within the application (e.g., fast or methodical), pattern of launching/closing of the application (e.g., whether the requestor opened and closed the application numerous times before submitting the ride request), time lapse between actions, and any other application usage data may correlate to the requestor's state of mind at the time the request was made. For example, if interaction with the application was fast and the request was made efficiently, it may indicate that the requestor was in a rush. If on the other hand, the interaction was slow and the requestor took a long time to make the request, it may indicate that the requestor was not in a rush. Such signals that conceptually may indicate whether the requestor is in a rush may assist the machine-learning model with predicting whether the requestor would appreciate soothing or exciting music, or no music at all. In particular embodiments, the transportation application may access other types of application usage data from other applications (including the operating system) installed on the requestor's device and use them as contextual information 715. This may include other applications that are being used before and/or after the ride was requested (e.g., whether it was a gaming application or productivity application). The transportation application may also query the other applications for data relating to the requestor (e.g., age, gender, circle of friends, the friends' interests, etc.). The transportation application may further obtain data such as, e.g., the requestor's location before or at the scheduled pickup time, the origination and destination locations, and any other ride information. Such information may indicate whether the requestor is going home from work, going to work from home, picking children up, running an errand, etc., which may also serve as contextual information 715.

Any of the aforementioned types of data (e.g., user profile information 711, contextual information 715, or any other data) may correlate with a ride requestor's preference for certain personalization features, and such correlation may be automatically learned by the machine-learning model 720. In particular embodiments, during training, the machine-learning model 720 may process the training data sample (e.g., user profile 711 and/or contextual information 715) and, based on the current parameters of the machine-learning model 720, generate a prediction 730. The type of prediction 730 generated may depend to the training label 719 associated with the training sample 710. For example, if the training label 719 indicates a particular type of known user preference (e.g., temperature preference and/or music preference), the machine-learning model 720 would learn to predict 730 similar types of user preferences (e.g., temperature preference and/or music preference) based on input data associated with a given user profile 711 and/or contextual information 715. In particular embodiments, during training, the generated prediction 730 and the training label 719 may be compared 740. For example, the comparison 740 may be based on a loss function that measures a difference between the generated prediction 730 and the training label 719. Based on the comparison 740 or the corresponding output of the loss function, a training algorithm may update the parameters of the machine-learning model 720, with the objective of minimizing the differences or loss between subsequent predictions 730 and the corresponding labels 719. By iteratively training in this manner, the machine-learning model 720 may “learn” from the different training data samples and become better at generating predictions 730 that are similar to the known “ground truth” represented by the training labels 719.

Returning to FIG. 6, at step 640, the transportation management system may determine the current locations of the ride requestor and the ride provider. In particular embodiments, the location information may be determined using the GPS capabilities of their respective devices. For instance, the transportation application installed on the requestor's device may access the device's geolocation and send the information to the transportation management system. The geolocation of the ride provider may be ascertained in a similar fashion. In embodiments where the ride provider is an autonomous vehicle, the transportation management system may directly query the vehicle for its geolocation, or such information may be constantly transmitted to the transportation management system.

At step 650, the transportation management system may determine whether the ride provider (e.g., traditional human-driver or an autonomous vehicle) is sufficiently close to the ride requestor and/or the scheduled pickup location/time. The metric for determining closeness may be based on, e.g., a threshold distance and/or travel time. The determination made in step 650 may be useful in determining when the personalization should take place. For example, while a human ride provider is en route to the pick-up location, it may not be necessary to prematurely personalize the vehicle configurations, since doing so may not be desirable to the ride provider. This may be less of a concern for autonomous vehicles since there would be no driver to inconvenience. However, certain personalization that requires resources (e.g., turning on the air conditioning, opening the window and creating drag, turning on audio or video, etc.), may unnecessarily waste such resources since no one is in the vehicle. Thus, to avoid inconveniencing the ride provider and/or wasting resources unnecessarily, the transportation management system may trigger the personalization features upon a determination that the ride requestor has been or is about to be picked up. In particular embodiments, it may be desirable for certain personalization to commence prior to picking up the ride requestor, such as informing the ride provider of certain preferences of the requestor and adjusting the temperature, seat configuration, and any other configuration that may take time to change.

As shown in FIG. 6, if the system determines that the ride provider is not sufficiently close to the ride requestor or target location/time, it may repeat step 640 until the ride provider is sufficiently close. Upon making such determination, the transportation management system may, at step 660, instruct the provider's communication device to actuate the personalization configurations. In particular embodiments, the provider's communication device may be a mobile device, a transportation management vehicle device placed in the vehicle (described in further detail below), or the vehicle itself. The instructions may cause the device receiving the instructions to configure the vehicle accordingly. For example, in particular embodiments, the provider's mobile device may be communicatively coupled to a transportation management vehicle device, which may be connected to the vehicle via a CANvas interface or any other interface that allow an external computing system to control and configure a vehicle. Through the transportation management vehicle device and the CANvas interface, for example, the ride provider's mobile device may relay or generate personalization instructions to the vehicle in accordance with the transportation management system's instructions. Alternatively, the transportation management system may instruct the transportation management vehicle device directly, which in turn may configure the vehicle via the CANvas interface. If the transportation management vehicle device is configured to communicate directly with the vehicle, the personalization instructions may be issued directly to the vehicle. As previously discussed, personalization may include, e.g., changing the entertainment content (e.g., audio, video, games, etc.), changing the temperature, opening the windows, reconfiguring the seats, and any other configuration changes supported by the vehicle. In particular embodiments, the personalization instructions may be directed to the provider's device and/or the transportation management vehicle device instead. For example, through either of those devices, the transportation management system may inform the ride provider of certain information about the ride requestor (e.g., via text, graphics, audio, or video), such as the ride requestor's preferred language, whether the ride requestor is in a rush, whether or not the ride requestor welcomes conversation, and any information that would help the ride provider accommodate the needs of the ride requestor.

In particular embodiments, the personalized ride configurations may last for the duration of the ride requestor's journey. At step 670, the transportation management system may periodically check whether the ride requestor has reached the ride requestor's destination. The transportation management system may make such determination by comparing whether the ride provider's location and/or the ride requestor's location coincides with the destination. The transportation management system may also make such determination based on a notification that the ride has terminated from the device of either the ride requestor or provider. For example, the device may receive an indication from its owner that the ride has terminated (e.g., the ride requestor may have gotten off the vehicle at a different location). Upon determining that the requested ride has ended, the transportation management system may, at step 680, instruct the ride provider's communication device (e.g., mobile computing device, transportation management vehicle device, and/or the vehicle) to revert the ride configurations back to a default state or to the state prior to personalization. In particular embodiments, prior to actuating a personalization, the pre-personalization state may be saved, in whole or in part, in the local storage of any of the mobile computing device, transportation management vehicle device, the vehicle, and/or the transportation management system. For example, after the ride requestor exits the vehicle, the music may be turned off, the windows rolled down, the temperature raised, and any other ride configuration that was personalized may be reverted to its default or pre-personalization state.

Particular embodiments may repeat one or more steps of the method of FIG. 6, where appropriate. Although this disclosure describes and illustrates particular steps of the method of FIG. 6 as occurring in a particular order, this disclosure contemplates any suitable steps of the method of FIG. 6 occurring in any suitable order. Moreover, although this disclosure describes and illustrates an example method for personalizing ride configurations, including the particular steps of the method of FIG. 6, this disclosure contemplates any suitable method for personalizing ride configurations, including any suitable steps, which may include all, some, or none of the steps of the method of FIG. 6, where appropriate. Furthermore, although this disclosure describes and illustrates particular components, devices, or systems carrying out particular steps of the method of FIG. 6, this disclosure contemplates any suitable combination of any suitable components, devices, or systems carrying out any suitable steps of the method of FIG. 6. For example, while the steps in FIG. 6 may be performed by the transportation management system, any combination of those steps may be performed by any other computing system, including, e.g., the ride requestor's computing device, the ride provider's computing device, the transportation management vehicle device, and/or the vehicle. For instance, at step 660, the instructions triggering personalization may be issued by the ride requestor's computing device. The requestor's mobile device, through NFC or Bluetooth, may inform one or more devices of the provider (e.g., the provider's mobile device, the transportation management vehicle device, and/or the vehicle) of the personalization desired, and the provider's device may, in turn, actuate the personalization, depending on what the provider is willing to offer.

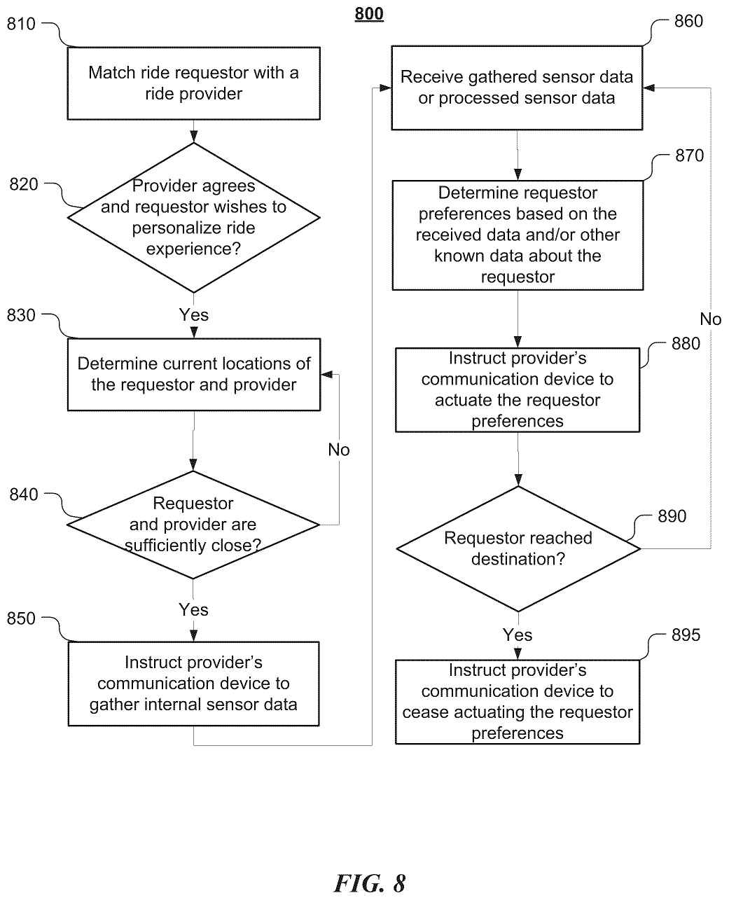

FIG. 8 illustrates an example of a method 800 for personalizing ride configurations based on real-time sensor data of the passenger compartment of a vehicle or other current contextual information. For example, even though a ride requestor may enjoy a variety of music, such as classical, rock-n-roll, pop, etc., certain music genre may be preferable over others depending on the requestor's mood, stress level, tasks, and any other current status or condition. Thus, in particular embodiments, sensors in the vehicle may be used to obtain sensor data associated with the passenger compartment of the vehicle, including any passenger therein, and the transportation management system may use the gathered data to determine the appropriate personalization configurations given the current context.

In particular embodiments, the method 800 may begin in a similar manner as described above with reference to FIG. 6, and for brevity would not be repeated in full. For example, a transportation management system may begin by matching a ride requestor with a ride provider (step 810), determining that the provider agrees and the requestor wishes to personalize certain ride configurations (step 820), and determining whether the current locations of the requestor and the provider are sufficiently close (step 830). Once it has been determined that the parties are sufficiently close (step 840), the transportation management system may proceed with personalizing the ride configurations of the vehicle (step 850).

At step 850, one or more of the provider's communication devices (e.g., mobile device, transportation management vehicle device, and the vehicle) may be instructed to begin gathering internal sensor data. In particular embodiments, the instructions may originate from the transportation management system, the ride requestor's mobile device, the ride provider's mobile device, or any of the other devices. For example, upon determining that the ride requestor and ride provider are sufficiently close, the transportation management system may send instructions to the ride provider's mobile device, which in turn may instruct the transportation management vehicle device and/or the vehicle to begin gathering internal sensor data. As another example, the transportation management system may send instructions directly to the sensors, the transportation management vehicle device, and/or the vehicle. In particular embodiments, the presence of the ride requestor's mobile device may trigger the sensors. For example, the requestor's mobile device, via short-range wireless communication (e.g., NFC, Bluetooth), may inform any of the ride provider's communication device (e.g., mobile device, transportation management vehicle device, and the vehicle) that the ride requestor has entered the vehicle, and therefore the sensors may begin gathering data.

In particular embodiments, the ride requestor, for privacy reasons, may opt-in or opt-out of allowing internal sensor data to be used for personalization. Similarly, the ride provider may opt-in or opt-out of offering personalization based on internal sensor data. The transportation management system and/or individual computing devices may store such preference indicators for the ride requestor and ride provider. In particular embodiments, the system's ride-matching process may try to connect ride requestors who desire personalization with ride providers who offer such service. In particular embodiments, the internal sensors may commence gathering data only with the consent of both the ride requestor and ride provider. In embodiments where the ride provider is an autonomous vehicle, only the ride requestor's consent may be needed.

In particular embodiments, sensors for capturing data within the passenger compartment of the vehicle may be located anywhere within the vehicle. For example, sensors may be located in, e.g., the vehicle's dashboard, rear-view mirror, ceiling, seats, door frame, and any other suitable location. In particular embodiments, sensors may also be integrated with the transportation management vehicle device, which is described in more detail herein elsewhere. The present disclosure contemplates any suitable sensor for gathering data relating to the passenger compartment of the vehicle. Sensors may include, for example, cameras, microphones, infrared sensors, sonars, LiDARs, and any other suitable sensors. In particular embodiments, the actuated sensors may also include those for gathering data relating to the environment external to the vehicle, such as weather, temperature, traffic condition, lighting, etc. Any of the sensor data, whether pertaining to the interior or exterior of the vehicle, may be used to predict the types of personalization that the ride requestor would appreciate.

At step 860, the transportation management system may receive the sensor data or processed sensor data through one or more devices within the vehicle (e.g., the provider's mobile device, the transportation management vehicle device, the vehicle, and/or the requestor's mobile device). In particular embodiments, the transportation management system may receive the raw sensor data obtained by the sensors or processed sensor data. In particular embodiments, the vehicle, the transportation management vehicle device, and/or the provider's computing device may be communicatively coupled to the sensors and may process the gathered data. Processed sensor data, for example, may include aggregate sensor data (e.g., average or statistical sensor data over a period of time), significant or sudden changes in sensor data (e.g., relative to a predetermined benchmark), conclusions or predictions drawn from the sensor data (e.g., the passenger is hot or cold, stressed or relaxed, etc.), and any other derivative information from the sensor data.

At step 870, the transportation management system may determine personalization preferences of the ride requestor based on any data available to the system. This may include the sensor data or processed sensor data, which may provide current contextual information, as well as any relevant user profile data, ride-sharing data, app usage data, or any other data relating to the ride requestor as described with reference to FIG. 6 and elsewhere herein. The current contextual information provided by the sensor data may be used to improve personalization predictions. Conceptually, for example, the sensors may capture signals from the ride requestors indicative of their mood, whether they are stressed or relaxed, whether they are occupied or engrossed in something or with someone, whether they feel hot or cold, etc. The contextual state of the ride requestor may be relevant to the requestor's current preferences. For example, if the requestor is stressed or in a bad mood, soothing music may be welcomed but not conversation with the driver; if the requestor is in a good mood, upbeat music may be appropriate rather than news; if the requestor is engaged in conversation, any distraction such as audio or video may be unwanted; and if the requestor is hot or cold, the vehicle's air conditioning unit may adjust the interior temperature. It should be appreciated that, in particular embodiments, the system may not need to determine the exact categorical context (e.g., mood, stress level, etc.) and/or how it correlates to particular personalization preferences. Rather, machine-learning may be used to learn the relationship between the sensor signals and personalization preferences. Such signals may include, for example, movement speed (e.g., based on video data capturing the speed in which the ride requestor opened the vehicle's door and entered the vehicle), the force used in closing the door (e.g., based on sound, speed, and vibration), the requestor's speech patterns (e.g., based on tone, pitch, rate), breathing pattern (e.g., based on sound), whether the requestor is engaged in conversation with another passenger or via a phone (e.g., based on voices, speech pattern, video data), the requestor's expression (e.g., based on images and voice data), attire or hair style (e.g., based on video data), body temperature (e.g., based on infrared sensor data), the outside weather or temperature (e.g., based on external light sensor, water sensor, and thermostat data), traffic congestion (e.g., based on ultrasound, LiDAR, odometer, and accelerometer data), and any other contextual signals that may be relevant to the ride requestor's personalization preference.

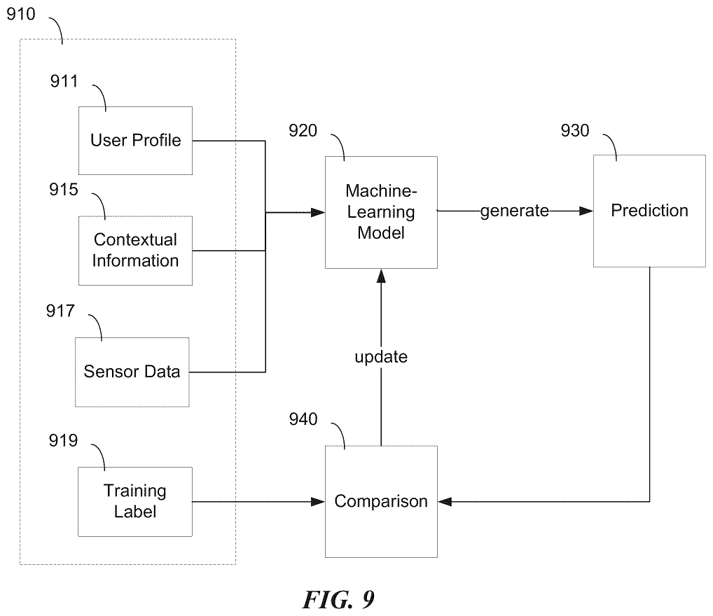

In particular embodiments, one or more machine-learning models may be trained to predict a given ride requestor's preferences. FIG. 9 illustrates an example block diagram for training a machine-learning model. For example, the machine-learning model 920 may be trained using a sufficiently large set of training data to learn to recognize features in the data that may signify or correlate to certain requestor preferences. Each training data sample 910 in the set of training data may be associated with a ride requestor and may be labeled 919 with known personalization preferences (e.g., preferred music genre, temperature, etc.) of that ride requestor in a given context, represented by sensor data 917 and/or other contextual data 915 captured at the time. In particular embodiments, the training data sample 910 representing that ride requestor may also include or be associated with other information known to the system, such as the requestor's profile data 911 (e.g., demographics information, ride-sharing history, etc.). These data, along with any other data relating to the ride requestor (e.g., third-party data) may be used to train the machine-learning model 920.

In particular embodiments, the machine-learning model 920 may learn to extract features from input data and generate predictions 930 that may, for example, represent predicted preferences of a ride requestor. The features may have meaningful interpretations (at least to humans), such as, for example, the ride requestor being cold (e.g., signified by the requestor shivering, putting on clothes, rubbing hands together, etc.), hot (e.g., signified by the requestor taking clothes off, self-fanning, panting, etc.), stressed (e.g., signified by the requestor frowning, sighing, nail-biting, etc.), busy (e.g., signified by the requestor flipping through paperwork, typing on laptop, talking on the phone, looking at the clock/watch, etc.), relaxed (e.g., signified by the ride requestor smiling, making conversation with the driver, nodding to the music, etc.), and tired (e.g., signified by closed eyes and/or head rested on the headrest, etc.). The features extracted by the machine-learning model 920 may also be unintelligible to humans and may simply represent data patterns that correlate with or tend to be present with certain ride-requestor preferences. Through training, the machine-learning model 920 may learn to identify predictive and non-predictive features and apply the appropriate weights to the features to optimize the model's 920 predictive accuracy. In embodiments where supervised learning is used and each training data sample 910 has a preference-type label 919, the training algorithm may iteratively process each training data sample 910 (including user profile data 911, contextual information 915, and/or sensor data 917) and generate a prediction 930 based on the model's 920 current parameters. The prediction 930 may be compared 940 with the preference-type label 919 of the training data sample 910 using, for example, a loss function that measures/quantifies the difference. Based on the comparison 940 results, the training algorithm may adjust the model's 920 parameters/configurations (e.g., weights) accordingly to minimize the differences between the generated predictions 930 and the corresponding labels 919. Any suitable machine-learning model and training algorithm may be used, including, e.g., neural networks, decision trees, clustering algorithms, and any other suitable machine-learning techniques. Once trained, the model 920 may take as input data associated with a ride requestor and output one or more predictions of what personalized configurations would likely be desirable to the ride requestor. The ride requestor and the vehicle associated with the input data may be different from any of the ride requestors and vehicles associated with the training data samples.

Returning to FIG. 8, at step 880, the transportation management system may send instructions configured to trigger personalization based on the determination made in step 870. In particular embodiments, the instructions may be sent to the provider's mobile device, the transportation management vehicle device, and/or the vehicle, and any of which may respond accordingly to actuate the desired personalization (e.g., the provider's mobile device may send corresponding instructions to the transportation management vehicle device, which in turn may configure the vehicle via a CANvas interface). As a result, various vehicle configurations may be personalized according to the predicted preferences of the ride requestor, thereby improving the requestor's overall ride experience.

In particular embodiments, while the ride requestor is still being transported in the vehicle, the transportation management system may continue to monitor the current sensor data to determine any updated contextual information. Thus, in the embodiment shown in FIG. 8, the transportation management system may continuously check, at step 890, whether the ride requestor has reached the ride requestor's destination or whether any other termination condition is satisfied (e.g., either the ride provider or the ride requestor indicates that the ride has terminated). If the ride is ongoing, the transportation management system may continue to receive sensor data or processed sensor data (step 860), determine any updated preferences (step 870), and cause personalization based on the determined preferences to be actuated (step 880). For example, based on the updated sensor data, the transportation management system may determine that the requestor is no longer hot and may, therefore, lower the strength of the air conditioning unit. As another example, the system may determine that the requestor has fallen asleep or is now calling someone, and as such, the system may respond by turning off or lowering the volume of any ongoing entertainment. Once the system determines that the ride requestor has reached the ride requestor's destination and exited the vehicle, the system may, at step 895, instruct the vehicle to revert to its default or pre-personalization configurations, as described with reference to FIG. 6.

In particular embodiments, the transportation management system may provide a translation service to facilitate communication between the ride provider and the ride requestor. For example, the system may use the process described above to determine what languages are spoken by the ride requestor and the ride provider once the ride requestor has been picked up. Using machine-learning models trained to detect languages spoken based on audible signals, the system may determine that the ride provider and ride requestor are speaking different languages. In particular embodiments, the parties' language proficiency may also be determined based on their respective profiles (e.g., each of them may indicate the languages they speak), which may be specified by the parties or learned by the transportation management system from their previous rides (e.g., in past rides, the transportation management system may have detected the requestor or provider speaking particular languages). Upon determining that the ride provider and ride requestor are speaking different languages or are not proficient in a common language, the transportation management system may invoke and configure an appropriate translation application to facilitate communication between the parties. The translation application may include, e.g., a text-based translation application installed on a device within the vehicle, or an audio-based translation application that may receive a spoken word in one language and output an audible corresponding word in another language. It should be appreciated that such techniques are known to one of ordinary skill in the art. The audible translation service may be provided via the transportation management vehicle device and/or the provider's computing device, for example.

In particular embodiments, the transportation management system may also provide productivity features. For example, the transportation application installed on the ride requestor's device may have been granted access to the ride requestor's task-management, calendaring, and/or email applications. As another example, the transportation management system may be given the requestor's credentials to third-party systems that host such information. In particular embodiments, the transportation management system may determine, based on the current sensor data and/or the requestor's ride origin and/or destination, that the ride requestor may appreciate being productive while riding in the vehicle. This determination may be made using machine-learning, as described elsewhere in this application. As such, the transportation management system may retrieve the ride requestor's tasks, calendar entries, and/or email from the third-party applications or servers and display them on an in-vehicle device (e.g., a tablet) or use text-to-speech technology to present the information audibly using, e.g., the transportation management vehicle device or any other device within the vehicle. In particular embodiments, if the requestor's task list includes a shopping list, the transportation management system may suggest a convenient stop-over location along the current route and/or provide visual routing suggestions to allow the requestor to decide whether or not to ask the ride provider for a detour to the store.

Particular embodiments may repeat one or more steps of the method of FIG. 8, where appropriate. Although this disclosure describes and illustrates particular steps of the method of FIG. 8 as occurring in a particular order, this disclosure contemplates any suitable steps of the method of FIG. 8 occurring in any suitable order. Moreover, although this disclosure describes and illustrates an example method for personalizing ride configurations, including the particular steps of the method of FIG. 8, this disclosure contemplates any suitable method for personalizing ride configurations, including any suitable steps, which may include all, some, or none of the steps of the method of FIG. 8, where appropriate. Furthermore, although this disclosure describes and illustrates particular components, devices, or systems carrying out particular steps of the method of FIG. 8, this disclosure contemplates any suitable combination of any suitable components, devices, or systems carrying out any suitable steps of the method of FIG. 8. For example, while the steps in FIG. 8 may be performed by the transportation management system, any combination of those steps may be performed by any other computing system, including, e.g., the ride requestor's computing device, the ride provider's computing device, the transportation management vehicle device, and/or the vehicle. For instance, at step 870, the ride provider's mobile, device, the transportation management vehicle device, or the vehicle may process the sensor data to determine the likely preferences of the ride requestor. In particular embodiments where machine-learning models are used for making such determination, the transportation management system may transmit a trained machine-learning model to the computing device in the vehicle to allow the determination to be made locally. This may be desirable since sensor data may be overly large to transmit to the remote transportation management system in a timely fashion. If the machine-learning model takes as input any other data (e.g., the requestor's profile data, ride-sharing data, etc.), such information may be made available to the computing device executing the machine-learning model (e.g., the computing device may obtain the data from the transportation management system and/or the ride requestor's mobile device). As another example, at step 880, the instructions triggering personalization may be issued by the ride requestor's computing device. The requestor's mobile device, through NFC or Bluetooth, may inform one or more devices of the provider (e.g., the provider's mobile device, the transportation management vehicle device, and/or the vehicle) of the personalization desired, and the provider's device may, in turn, actuate the personalization, depending on what the provider is willing to accommodate.

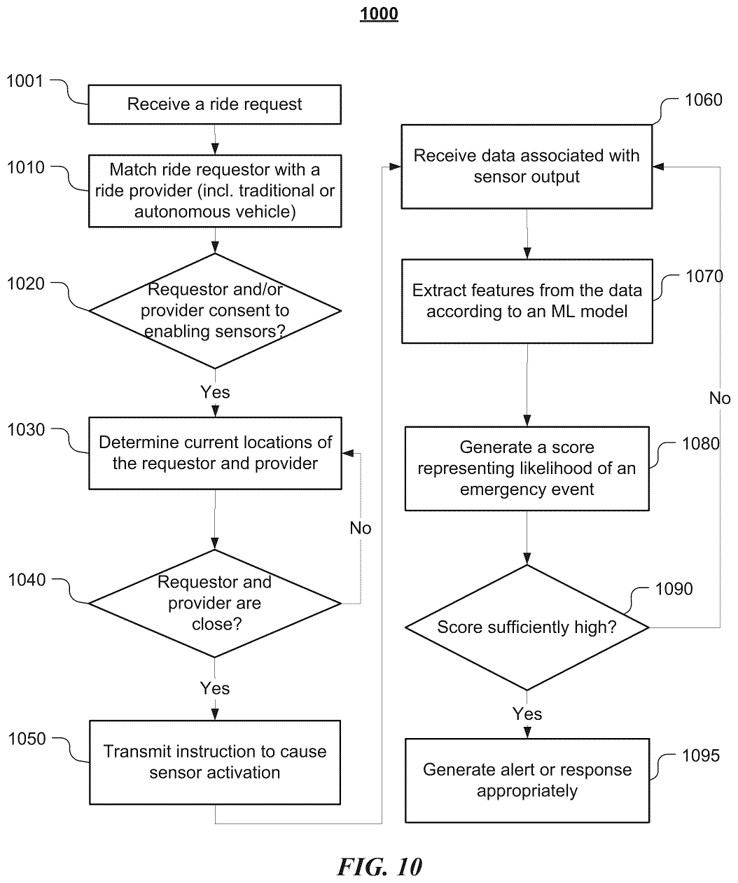

FIG. 10 illustrates an example of a method for detecting and responding to emergencies or other actionable events/incidents occurring within the passenger compartment of a vehicle. Sensors in the provider's vehicle may gather contextual information within the passenger compartment of the vehicle, and such information may be used to detect emergency situations that may warrant an immediate response. For example, in either conventional human-driven vehicles or autonomous vehicles, emergency situations may arise, such as a confrontation (e.g., between the ride requestor and any other passenger, including the driver) and health emergencies (e.g., heart attack, women going into labor, etc.). Additionally, other behavior that may violate ridership rules of the transportation management system may also be detected, such as inappropriate behavior and/or damaging the vehicle. In response to these detected events, the transportation management system may trigger any type of appropriate responses. For example, in response to detected improper behavior, the transportation management system may cause a verbal warning to be announced within the vehicle (e.g., via any of the computing devices within the vehicle that is communicatively connected to the transportation management system), which may further ask passengers in the vehicle to confirm the detected situation (e.g., if any of the passengers confirm that the improper behavior is occurring, the situation may be escalated). The transportation management system may also instruct an autonomous vehicle to pull over and unlock the doors, drive to the nearest emergency response facility (e.g., police station, fire department, etc.), and/or inform an emergency response team (e.g., police, firefighters, etc.) of the incident.

As another example, if a health emergency is detected, a remote health operator may be communicatively connected to the audiovisual system within the vehicle so that the health operator may assess the situation. In particular embodiments, the transportation management system may also call an ambulance and/or instruct the human driver (if the driver is still able to operate) or the autonomous vehicle to drive to the nearest hospital. As yet another example, if the detected behavior of any passenger violates any rules, the transportation management system may issue an audible warning within the autonomous vehicle, and/or flag the vehicle so that it is inspected for damage at its next inspection.

Turning to the embodiment shown in FIG. 10, at step 1001, a transportation management system may receive a ride request from a device associated with a ride requestor. The ride request may specify an origination location and a destination location, as well as other information as described elsewhere herein. At step 1010, the transportation management system may match a ride requestor with a ride provider, which may be a conventional human-operated vehicle or an autonomous vehicle, for transporting the ride requestor. The matching process may be performed in accordance with embodiments described herein, such as in the manner described with reference to FIG. 6.

At step 1020, the transportation management system may determine whether the requisite party or parties consent to activating one or more sensors in the vehicle while the ride requestor is in the vehicle. Since detection of events occurring within the vehicle involves using sensors to monitor activities within the vehicle, the system may optionally provide its users with the option to enable or disable the sensors to respect their privacy. The system may only require the consent of the ride provider, only require the consent of the ride requestor, or require the mutual consent of both the ride provider and the ride requestor. Naturally, if the ride provider is an autonomous vehicle, no consent would be necessary or expected. In particular embodiments, consent may be expressed during the current ride transaction. For example, ride requestors may indicate whether they consent when sending the ride request (e.g., the requestor's consent preferences may be stored on the device of the ride requestor or the requestor may at the time of the request specify through a user interface whether consent is granted). Similarly, the ride provider (if human) may indicate whether consent is granted when the ride provider accepts the ride request. The ride provider and ride requestor may also pre-store their respective consent preferences with the transportation management system (e.g., consent preference may be part of a user's profile information). Although the embodiment illustrated in FIG. 10 shows the transportation management system checking for consent (step 1020) after matching the ride requestor with the ride provider (step 1010), other embodiments may integrate consent checking with the matching process. The matching process in such embodiments may thus take into consideration whether a ride requestor and a ride provider both consent to the emergency-response feature, if mutual consent is required.

If the system determines that the consent requirement is met, the system at step 1030 may determine the current locations of the requestor and the provider, and at step 1040 determine whether they are sufficiently close to actuate the feature. If the requestor and provider are not sufficiently close (e.g., relative to a threshold proximity), the system may repeat step 1030 until the parties are sufficiently close before actuating the feature. The location of the ride requestor may be obtained from the device of the ride requestor, which may ascertain its current location using a GPS or other geo-positioning technology. Similarly, the location of the ride provider may be obtained from the ride provider's device if the ride provider is a human driver, or from the vehicle or the transportation management vehicle device. The transportation management system may compute a proximity measure between the two locations and determine whether the proximity satisfies predetermined criteria (e.g., coinciding location or within x feet or y minutes of travel time apart). In particular embodiments, the actuation of the feature may alternatively or additionally be triggered by the requestor's device establishing a short-range wireless connection (e.g., NFC, Bluetooth, Zigbee, etc.) with any of the devices within the vehicle.

At step 1050, once it has been determined that the parties are sufficiently close, the transportation management system may send instructions configured to cause sensors in the vehicle to be activated, so that they may begin sensing and generating sensor output (e.g., audio, video, LiDAR data, infrared reading, temperature measure, etc.). In particular embodiments, the instruction may be sent to the device(s) of the requestor and/or provider, the transportation management vehicle device, and/or the vehicle. Any of the recipients of the instruction may actuate the sensors directly or indirectly. For example, if a sensor is integrated with or under the direct control the vehicle or the transportation management vehicle device, issuing an instruction to the vehicle or the transportation management vehicle device may cause the sensor to be activated directly. However, if the transportation management system is unable to send instructions directly to the vehicle or the transportation management vehicle device, it may nevertheless indirectly activate the sensor. For example, to activate sensors that are integrated with the transportation management vehicle device, which may be directly controlled by the ride provider's mobile device (e.g., smartphone), the transportation management system may send the instruction to the provider's mobile device, which may in turn instruct the transportation management vehicle device to activate its sensor. In configurations where the transportation management vehicle device is able to control the vehicle to activate its sensor, the transportation management vehicle device may cause the vehicle to do so in response to instructions from the provider's mobile device. One of ordinary skill in the art would recognize that any communication path may exist between the devices in the vehicle (e.g., the provider's device, the requestor's device, the transportation management vehicle device, and the vehicle), and therefore the transportation management system may send instructions to any of the devices capable of receiving such instructions to trigger sensor activation.

At step 1060, the transportation management system may receive data associated with sensory output(s) from one or more sensors in the vehicle. Any type of sensor may be used to gather data pertaining to the passenger compartment of the vehicle. A sensory output may be, for example, images, videos, audios, LiDAR measures, infrared measures, temperature measures, GPS data, or any other information measured or detected by sensors. In particular embodiments, a sensory output may be the result of one or more sensors capturing environmental information associated with the passenger compartment of the vehicle, which may include the ride requestor, ride provider (if human), and any other passengers. For example, sensory output may be associated with at least the ride requestor while the ride requestor is at least partially within a passenger compartment of the vehicle (e.g., as the ride requestor is entering the vehicle or seated in the vehicle). In particular embodiments, data associated with sensory output may include the raw sensory output (e.g., the images, videos, audios, etc.) and/or data derived from processing the sensory output. For example, any of the devices within the vehicle (e.g., the vehicle's computer system, the transportation management vehicle device, the device of the ride provider or requestor, etc.) may process the sensory data and generate derivative data. For example, derivative data from audio may include data representing pitch, tone, speech rate, and/or semantic information. As another example, derivative data from image, video or LiDAR data may include data representing identified objects, motion, movement speed, gestures, etc. The transportation management system may receive any data associated with the sensory output from sensors, including raw sensory output and/or any derivative data.

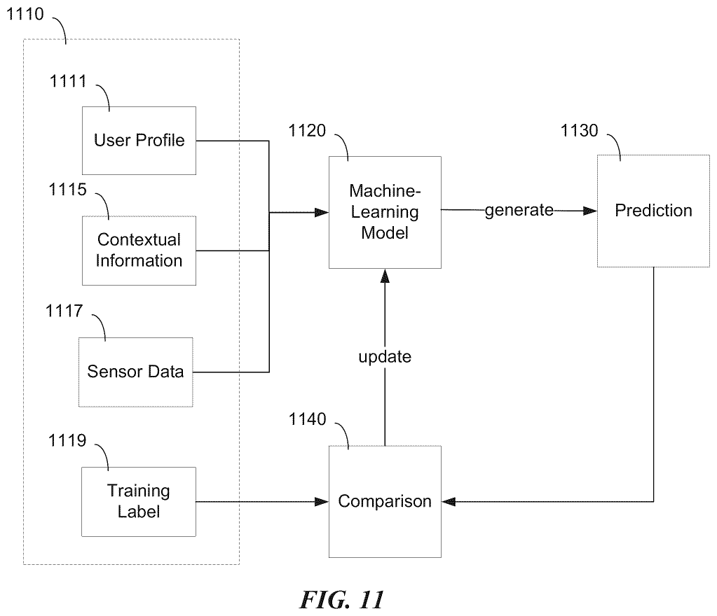

In particular embodiments, the transportation management system may process the received data and identify any actionable event of interest using a machine-learning model, trained using a set of training data. FIG. 11 illustrates an example block diagram for training a machine-learning model 1120 used for predicting emergencies or other actionable events/incidents (e.g., confrontations, health emergencies, etc.) occurring within the passenger compartment of a vehicle. The set of training data, in particular embodiments, may be created from data obtained from a fleet of vehicles managed by the transportation management system, including conventional human-driven vehicles and/or autonomous vehicles. Each training data sample obtained may include sensory data 1117, as well as the data derived therefrom, from sensors in vehicles that monitor the vehicles' respective passenger compartments. In particular embodiments, data 1117 from each vehicle that is associated with a time segment (e.g., 5, 10, 30, or 60 seconds) may be labeled 1119 or associated with one or more predetermined event types to represent what transpired or occurred in the associated vehicle during that time segment. For example, a particular training data sample 1110 capturing a health emergency occurring in a vehicle may be labeled 1119 or associated with a “health emergency” event type. Other event types may include, e.g., normal event (e.g., a regular passenger transportation event that requires no action), confrontation/argument, particular types of improper behavior that are against policy (e.g., vandalism, damaging the vehicle, smoking in a non-smoking vehicle, etc.), and any other event types that warrant action. In particular embodiments, the training data sample 1110 may also include user profile 1111 or other data related to those involved in the event (e.g., profile information 1111 of the ride provider, ride requestor, and/or any other passengers). For example, a particular training data sample 1110 may pertain to a particular incident that occurred in the past. The training data sample 1110 may be labeled 1119 or associated with the “improper behavior” event type, and the data 1110 may include audiovisual data 1117 of the event as it was occurring, along with profile data 1111 of the persons involved (e.g., the ride provider and/or requestor). In particular embodiments, the training data sample 1110 may further include contextual information 1115, such as the time of day of the ride, the weather, app-usage patterns associated with the ride request, etc., as previously described.

Using the training data, a machine-learning model 1120 may be trained so that it recognizes features of input data that signify or correlate to certain event types. For example, a trained machine-learning model 1120 may recognize data features that signify the likelihood of an emergency situation, improper behavior, or any other types of actionable event. In particular embodiments, the features may have meaningful interpretations (at least to humans), such as bad language, pitch, tone, excessive physical movements, etc. As another example, features that correlate to a health emergency may include signals that conceptually represent, e.g., a person laying down, shallow breathing, blood, seizure spasms, audible or gestured request for help, etc. The features may also be unintelligible to humans and may simply represent data patterns that tend to be present when certain event types occur. Through training, the machine-learning model 1120 may learn to identify predictive and non-predictive features and apply the appropriate weights to the features to optimize the model's 1120 predictive accuracy. In embodiments where supervised learning is used and each training data sample 1110 has an event-type label 1119, the training algorithm may iteratively process each training data sample 1110 (including user profile data 1111, contextual information 1115, and/or sensor data 1117), and generate a prediction 1130 based on the model's 1120 current parameters. The prediction 1130 may be compared 1140 with the event-type label 1119 of the training data sample 1110 using, for example, a loss function that measures/quantifies the difference. Based on the comparison 1140 results, the training algorithm may adjust the model's 1120 parameters/configurations (e.g., weights) accordingly to minimize the differences between the generated predictions 1130 and the corresponding labels 1119. Any suitable machine-learning model and training algorithm may be used, including, e.g., neural networks, decision trees, clustering algorithms, and any other suitable machine-learning techniques. Once trained, the model 1120 may take as input data associated with a ride requestor and output one or more predictions that indicate a likelihood that an actionable event has occurred. The ride requestor and the vehicle associated with the input data may be different from any of the ride requestors and vehicles associated with the training data samples.

Returning to FIG. 10, at step 1070, the transportation management system may extract features from the received data according to a machine-learning model. The machine-learning model is able to automatically do so based on what it learned during the training process. In particular embodiments, appropriate weights that were learned during the training process may be applied to the features. At step 1080, the machine-learning model, based on the features of the received data, may generate a score representing a likelihood or confidence that the received data is associated with a particular event type (e.g., improper behavior, confrontation, health emergency, etc.).

At 1090, the transportation management system may determine whether the score is sufficiently high relative to a threshold or criteria to warrant certain action. If the score is not sufficiently high, thus indicating that the detected event may not have actually occurred (in other words, a false-positive), the system may return to step 1060 and continue to monitor subsequent incoming data. On the other hand, if the score is sufficiently high, then at step 1095 the system may generate an appropriate alert and/or determine an appropriate action/response. In particular embodiments, the system may send alerts to appropriate recipients based on the detected event types. For instance, an alert of illegal activity may be sent to a third-party responder such as a private security dispatch center or the local police; an alert of health emergency may be sent to a nearby hospital, ambulatory service, and/or firefighters; an alert of improper or restricted activities (e.g., eating/drinking, etc.) may be sent to an internal agency associated with the system that handles such matters; and an alert of vandalism may be sent to an internal agency as well as a service center database so that the vehicle may be inspected/repaired when it is being serviced. An alert may additionally or alternatively be sent to one of the devices within the vehicle in which the event is occurring. For example, the alert may warn the passengers that events in the vehicle are being recorded, inform the passengers that an appropriate third-party has been contacted (e.g., police, ambulance, etc.), or provide guidance on what to do (e.g., remain calm in the vehicle, exit the vehicle, etc.). In situations where the vehicle is an autonomous vehicle, the system may also instruct an autonomous vehicle to perform certain actions. For example, the system may determine an alternative destination different from the originally intended destination and instruct the autonomous vehicle to drive to that alternative destination. For instance, the vehicle may be instructed to drive to the nearest hospital in the event of a health emergency or drive to the nearest police station in the event of a physical confrontation or illegal activity. In particular embodiments, the system may also instruct the vehicle to perform other actions, such as pulling over or increasing/reducing speed in the event of a health emergency.

Particular embodiments may repeat one or more steps of the method of FIG. 10, where appropriate. Although this disclosure describes and illustrates particular steps of the method of FIG. 10 as occurring in a particular order, this disclosure contemplates any suitable steps of the method of FIG. 10 occurring in any suitable order. Moreover, although this disclosure describes and illustrates an example method for detecting and responding to in-ride events, including the particular steps of the method of FIG. 10, this disclosure contemplates any suitable method for detecting and responding to in-ride events, including any suitable steps, which may include all, some, or none of the steps of the method of FIG. 10, where appropriate. Furthermore, although this disclosure describes and illustrates particular components, devices, or systems carrying out particular steps of the method of FIG. 10, this disclosure contemplates any suitable combination of any suitable components, devices, or systems carrying out any suitable steps of the method of FIG. 10. For example, while the steps in FIG. 10 may be performed by the transportation management system, any combination of those steps may be performed by any other computing system, including, e.g., the ride requestor's computing device, the ride provider's computing device, the transportation management vehicle device, and/or the vehicle. For instance, from step 1060 to 1090, the ride provider's mobile device, the transportation management vehicle device, or the vehicle may process the sensor data to detect any event of interest. In particular embodiments where machine-learning models are used for making such determination, the transportation management system may transmit a trained machine-learning model to the computing system in the vehicle to allow event-detection to be made locally. This may be desirable since sensor data may be overly large to transmit to the remote transportation management system in a timely fashion. If the machine-learning model takes as input other non-local data (e.g., the requestor's profile data, ride-sharing data, etc.), such information may be made available to the computing device executing the machine-learning model (e.g., the computing device may obtain the data from the transportation management system, the ride provider's device, or the ride requestor's device). In particular embodiments, the local computing device may send the event-detection determination to the transportation management system and let it perform the appropriate actions (e.g., generate alerts, etc., as described with reference to step 1095).

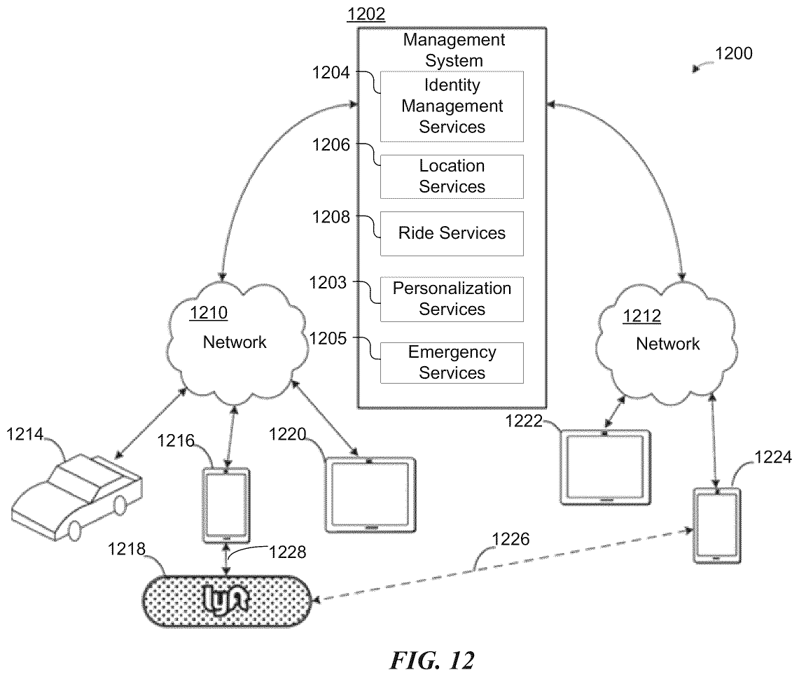

FIG. 12 shows a transportation management environment 1200, in accordance with particular embodiments. For example, a transportation management system 1202 executing on one or more servers or distributed systems may be configured to provide various services to ride requestors and providers. In particular embodiments, the transportation management system 1202 may include software modules or applications, including, e.g., identity management services 1204, location services 1206, ride services 1208, personalization services 1203, emergency services 1205, and/or any other suitable services. Although a particular number of services are shown as being provided by system 1202, more or fewer services may be provided in various embodiments. In addition, although these services are shown as being provided by the system 1202, all or a portion of any of the services may be processed in a distributed fashion. For example, computations associated with a service task may be performed by a combination of the transportation management system 1202 (including any number of servers, databases, etc.), one or more devices associated with the provider (e.g., devices integrated with the managed vehicles 1214, provider's computing devices 1216 and tablets 1220, and transportation management vehicle devices 1218), and/or one or more devices associated with the ride requestor (e.g., the requestor's computing devices 1224 and tablets 1222). In particular embodiments, the transportation management system 1202 may include one or more general purpose computers, server computers, distributed computing systems, clustered computing systems, cloud-based computing systems, or any other computing systems or arrangements of computing systems. The transportation management system 1202 may be configured to run any or all of the services and/or software applications described herein. In particular embodiments, the transportation management system 1202 may include an appropriate operating system as well as various server applications, such as web servers capable of handling hypertext transport protocol (HTTP) requests, file transfer protocol (FTP) servers, database servers, etc.

In particular embodiments, identity management services 1204 may be configured to, e.g., perform authorization services for requestors and providers and manage their interactions and data with the transportation management system 1202. This may include, e.g., authenticating the identity of providers and determining that they are authorized to provide services through the transportation management system 1202. Similarly, requestors' identities may be authenticated to determine whether they are authorized to receive the requested services through the transportation management system 1202. Identity management services 1204 may also manage and control access to provider and requestor data maintained by the transportation management system 1202, such as driving and/or ride histories, vehicle data, personal data, preferences, usage patterns as a ride provider and as a ride requestor, profile pictures, linked third-party accounts (e.g., credentials for music or entertainment services, social-networking systems, calendar systems, task-management systems, etc.) and any other associated information. The management service 1204 may also manage and control access to provider/requestor data stored with and/or obtained from third-party systems. For example, a requester or provider may grant the transportation management system 1202 access to a third-party email, calendar, or task management system (e.g., via the user's credentials). As another example, a requestor or provider may grant, through a mobile device (e.g., 1216, 1220, 1222, and 1224), a transportation application associated with the transportation management system 1202 access to data provided by other applications installed on the mobile device. Such data may be processed on the client and/or uploaded to the transportation management system 1202 for processing, if so desired.

In particular embodiments, the transportation management system 1202 may provide location services 1206, which may include navigation and/or traffic management services and user interfaces. For example, the location services 1206 may be responsible for querying devices associated with the provider (e.g., vehicle 1214, computing device 1216, tablet 1220, transportation management vehicle device 1218) and the requester (e.g., computing device 1224 and tablet 1222) for their locations. The location services 1206 may also be configured to track those devices to determine their relative proximities, generate relevant alerts (e.g., proximity is within a threshold distance), generate navigation recommendations, and any other location-based services.

In particular embodiments, the transportation management system 1202 may provide ride services 1208, which may include ride matching and management services to connect a requestor to a provider. For example, after the identity of a ride requestor has been authenticated by the identity management services module 1204, the ride services module 1208 may attempt to match the requestor with one or more ride providers. In particular embodiments, the ride services module 1208 may identify an appropriate provider using location data obtained from the location services module 1206. The ride services module 1208 may use the location data to identify providers who are geographically close to the requestor (e.g., within a certain threshold distance or travel time) and further identify those who are a good match with the requestor. The ride services module 1208 may implement matching algorithms that score providers based on, e.g.: preferences of providers and requestors; vehicle features, amenities, condition, and status; provider's preferred general travel direction, range of travel, and availability; requestor's origination and destination locations, time constraints, and vehicle feature needs; and any other pertinent information for matching requestors with providers. In particular embodiments, the ride services 1208 may use rule-based algorithms or machine-learning models for matching requestors and providers.

In particular embodiments, the personalization services 1203 may be responsible for personalizing a ride experience for a requestor. The personalization services 1203 may retrieve and/or predict ride preferences of the requestor using data associated with the requestor, including, e.g., the requestor's profile data, ride-sharing data, current contextual data, and any other suitable data related to the requestor. Such data may be obtained using the identity management services 1204, location services 1206, and ride services 1208, as well as from the requestor's computing device 1224 and tablet 1222, the provider's computing device 1216 and tablet 1220, the transportation management vehicle device 1218, and the vehicle 1214. In particular embodiments, the personalization services 1203 may use rule-based algorithms or machine-learning models to predict the types of ride configurations that would likely be appreciated by the requestor.

In particular embodiments, the emergency services 1205 may be responsible for detecting an emergency or other event types occurring in the passenger compartment of a vehicle 1214 and react accordingly. For example, the emergency services 1205 may obtain data associated with the passenger compartment, such as sensor data, via the vehicle 1214, the ride-service device 1218, the provider's computing devices 1216 and 1220, and the requestor's computing devices 1222 and 1224. Using data relating to occurrences in the passenger compartment, together with data known about the provider, requestor, and the ride, the emergency services 1205 may generate a risk factor score that indicates a likelihood of an emergency situation (e.g., improper behavior, health emergency, etc.). Based on the risk factor score, the emergency services 1205 may generate and send an alert to the appropriate agencies, such as the police, firefighters, ambulance dispatch centers, etc. Where appropriate, the emergency services 1205 may send alerts to the vehicle 1214, ride-service device 1218, provider devices 1216 and 1220, and requestor devices 1222 and 1224. The alerts may inform the passengers of the nature of the detected emergency, what has been done in response (e.g., police being contacted), provide appropriate instructions (e.g., directions to a hospital), warnings (e.g., cease the improper behavior), and any other appropriate responses. In particular embodiments, the alerts may also cause the vehicle 1214 (e.g., via the ride-service device 1218 or to the vehicle 1214 directly) to stop or pull over.

The transportation management system 1202 may communicatively connect to various devices through networks 1210 and 1212. Networks 1210, 1212 may include any combination of interconnected networks configured to send and/or receive data communications using various communication protocols and transmission technologies. In particular embodiments, networks 1210, 1212 may include local area networks (LAN), wide-area network, and/or the Internet, and may support communication protocols such as transmission control protocol/Internet protocol (TCP/IP), Internet packet exchange (IPX), systems network architecture (SNA), and any other suitable network protocols. In particular embodiments, data may be transmitted through networks 1210, 1212 using a mobile network (such as a mobile telephone network, cellular network, satellite network, or other mobile network), PSTNs (a public switched telephone networks), wired communication protocols (e.g., USB, CAN, CANvas), and/or wireless communication protocols (e.g., WLAN technologies implementing the IEEE 802.11 family of standards, Bluetooth, Bluetooth Low Energy, NFC, Z-Wave, and ZigBee). In particular embodiments, networks 1210, 1212 may each include any combination of networks described herein or known to one of ordinary skill in the art.

In particular embodiments, devices within a vehicle may be interconnected. For example, any combination of the following may be communicatively connected: vehicle 1214, provider computing device 1216, provider tablet 1220, transportation management vehicle device 1218, requestor computing device 1224, requestor tablet 1222, and any other device (e.g., smart watch, smart tags, etc.). For example, the transportation management vehicle device 1218 may be communicatively connected to the provider computing device 1216 and/or the requestor computing device 1224. The transportation management vehicle device 1218 may connect 1226, 1228 to those devices via any suitable communication technology, including, e.g., WLAN technologies implementing the IEEE 802.11 family of standards, Bluetooth, Bluetooth Low Energy, NFC, Z-Wave, ZigBee, and any other suitable short-range wireless communication technology.

In particular embodiments, users may utilize and interface with one or more services provided by the transportation management system 1202 using applications executing on their respective computing devices (e.g., 1214, 1216, 1218, and/or 1220), which may include mobile devices (e.g., an iPhone®, an iPad®, mobile telephone, tablet computer, a personal digital assistant (PDA)), laptops, wearable devices (e.g., smart watch, smart glasses, head mounted displays, etc.), thin client devices, gaming consoles, and any other computing devices. In particular embodiments, provider computing device 1214 may include a vehicle-integrated computing device, such as a vehicle navigation system, or other computing device integrated with the vehicle itself, such as the management system of an autonomous vehicle. The computing device may run on any suitable operating systems, such as Android®, iOS®, macOS®, Windows®, Linux®, UNIX®, or UNIX®-based or Linux®-based operating systems, or other operating systems. The computing device may further be configured to send and receive data over the Internet, short message service (SMS), email, and various other messaging applications and/or communication protocols. In particular embodiments, one or more software applications may be installed on the computing device of a provider or requestor, including an application associated with the transportation management system 1202. The transportation application may, for example, be distributed by an entity associated with the transportation management system via any distribution channel, such as an online source from which applications may be downloaded and/or via physical media, such as CDs and DVDs. Additional third-party applications unassociated with the transportation management system may also be installed on the computing device. In particular embodiments, the transportation application may communicate or share data and resources with one or more of the installed third-party applications.

FIG. 13 illustrates an example block diagram of a transportation management environment for matching ride requestors with autonomous vehicles. In particular embodiments, the environment may include various computing entities, such as a user computing device 1330 of a user 1301 (e.g., a ride provider or requestor), a transportation management system 1360, an autonomous vehicle 1340, and one or more third-party system 1370. The computing entities may be communicatively connected over any suitable network 1310. As an example and not by way of limitation, one or more portions of network 1310 may include an ad hoc network, an extranet, a virtual private network (VPN), a local area network (LAN), a wireless LAN (WLAN), a wide area network (WAN), a wireless WAN (WWAN), a metropolitan area network (MAN), a portion of the Internet, a portion of Public Switched Telephone Network (PSTN), a cellular network, or a combination of any of the above. In particular embodiments, any suitable network arrangement and protocol enabling the computing entities to communicate with each other may be used. Although FIG. 13 illustrates a single user device 1330, a single transportation management system 1360, a single vehicle 1340, a plurality of third-party systems 1370, and a single network 1310, this disclosure contemplates any suitable number of each of these entities. As an example and not by way of limitation, the network environment may include multiple users 1301, user devices 1330, transportation management systems 1360, autonomous-vehicles 1340, third-party systems 1370, and networks 1310.

The user device 1330, transportation management system 1360, autonomous vehicle 1340, and third-party system 1370 may be communicatively connected or co-located with each other in whole or in part. These computing entities may communicate via different transmission technologies and network types. For example, the user device 1330 and the vehicle 1340 may communicate with each other via a cable or short-range wireless communication (e.g., Bluetooth, NFC, WI-FI, etc.), and together they may be connected to the Internet via a cellular network that is accessible to either one of the devices (e.g., the user device 1330 may be a smartphone with LTE connection). The transportation management system 1360 and third-party system 1370, on the other hand, may be connected to the Internet via their respective LAN/WLAN networks and Internet Service Providers (ISP). FIG. 13 illustrates transmission links 1350 that connect user device 1330, autonomous vehicle 1340, transportation management system 1360, and third-party system 1370 to the communication network 1310. This disclosure contemplates any suitable transmission links 1350, including, e.g., wire connections (e.g., USB, Lightning, Digital Subscriber Line (DSL) or Data Over Cable Service Interface Specification (DOCSIS)), wireless connections (e.g., WI-FI, WiMAX, cellular, satellite, NFC, Bluetooth), optical connections (e.g., Synchronous Optical Networking (SONET), Synchronous Digital Hierarchy (SDH)), any other wireless communication technologies, and any combination thereof. In particular embodiments, one or more links 1350 may connect to one or more networks 1310, which may include in part, e.g., ad-hoc network, the Intranet, extranet, VPN, LAN, WLAN, WAN, WWAN, MAN, PSTN, a cellular network, a satellite network, or any combination thereof. The computing entities need not necessarily use the same type of transmission link 1350. For example, the user device 1330 may communicate with the transportation management system via a cellular network and the Internet, but communicate with the autonomous vehicle 1340 via Bluetooth or a physical wire connection.

In particular embodiments, the transportation management system 1360 may fulfil ride requests for one or more users 1301 by dispatching suitable vehicles. The transportation management system 1360 may receive any number of ride requests from any number of ride requestors 1301. In particular embodiments, a ride request from a ride requestor 1301 may include an identifier that identifies the ride requestor in the system 1360. The transportation management system 1360 may use the identifier to access and store the ride requestor's 1301 information, in accordance with the ride requestor's privacy settings. The ride requestor's 1301 information may be stored in one or more data stores (e.g., a relational database system) associated with and accessible to the transportation management system 1360. In particular embodiments, ride requestor information may include profile information about a particular ride requestor 1301. In particular embodiments, the ride requestor 1301 may be associated with one or more categories or types, through which the ride requestor 1301 may be associated with aggregate information about certain ride requestors of those categories or types. Ride information may include, for example, preferred pick-up and drop-off locations, driving preferences (e.g., safety comfort level, preferred speed, rates of acceleration/deceleration, safety distance from other vehicles when travelling at various speeds, route, etc.), entertainment preferences and settings (e.g., preferred music genre or playlist, audio volume, display brightness, etc.), temperature settings, whether conversation with the driver is welcomed, frequent destinations, historical riding patterns (e.g., time of day of travel, starting and ending locations, etc.), preferred language, age, gender, or any other suitable information. In particular embodiments, the transportation management system 1360 may classify a user 1301 based on known information about the user 1301 (e.g., using machine-learning classifiers), and use the classification to retrieve relevant aggregate information associated with that class. For example, the system 1360 may classify a user 1301 as a young adult and retrieve relevant aggregate information associated with young adults, such as the type of music generally preferred by young adults.

Transportation management system 1360 may also store and access ride information. Ride information may include locations related to the ride, traffic data, route options, optimal pick-up or drop-off locations for the ride, or any other suitable information associated with a ride. As an example and not by way of limitation, when the transportation management system 1360 receives a request to travel from San Francisco International Airport (SFO) to Palo Alto, California, the system 1360 may access or generate any relevant ride information for this particular ride request. The ride information may include, for example, preferred pick-up locations at SFO; alternate pick-up locations in the event that a pick-up location is incompatible with the ride requestor (e.g., the ride requestor may be disabled and cannot access the pick-up location) or the pick-up location is otherwise unavailable due to construction, traffic congestion, changes in pick-up/drop-off rules, or any other reason; one or more routes to navigate from SFO to Palo Alto; preferred off-ramps for a type of user; or any other suitable information associated with the ride. In particular embodiments, portions of the ride information may be based on historical data associated with historical rides facilitated by the system 1360. For example, historical data may include aggregate information generated based on past ride information, which may include any ride information described herein and telemetry data collected by sensors in autonomous vehicles and/or user devices. Historical data may be associated with a particular user (e.g., that particular user's preferences, common routes, etc.), a category/class of users (e.g., based on demographics), and/or all users of the system 1360. For example, historical data specific to a single user may include information about past rides that particular user has taken, including the locations at which the user is picked up and dropped off, music the user likes to listen to, traffic information associated with the rides, time of the day the user most often rides, and any other suitable information specific to the user. As another example, historical data associated with a category/class of users may include, e.g., common or popular ride preferences of users in that category/class, such as teenagers preferring pop music, ride requestors who frequently commute to the financial district may prefer to listen to news, etc. As yet another example, historical data associated with all users may include general usage trends, such as traffic and ride patterns. Using historical data, the system 1360 in particular embodiments may predict and provide ride suggestions in response to a ride request. In particular embodiments, the system 1360 may use machine-learning, such as neural networks, regression algorithms, instance-based algorithms (e.g., k-Nearest Neighbor), decision-tree algorithms, Bayesian algorithms, clustering algorithms, association-rule-learning algorithms, deep-learning algorithms, dimensionality-reduction algorithms, ensemble algorithms, and any other suitable machine-learning algorithms known to persons of ordinary skill in the art. The machine-learning models may be trained using any suitable training algorithm, including supervised learning based on labeled training data, unsupervised learning based on unlabeled training data, and/or semi-supervised learning based on a mixture of labeled and unlabeled training data.

In particular embodiments, transportation management system 1360 may include one or more server computers. Each server may be a unitary server or a distributed server spanning multiple computers or multiple datacenters. The servers may be of various types, such as, for example and without limitation, web server, news server, mail server, message server, advertising server, file server, application server, exchange server, database server, proxy server, another server suitable for performing functions or processes described herein, or any combination thereof. In particular embodiments, each server may include hardware, software, or embedded logic components or a combination of two or more such components for carrying out the appropriate functionalities implemented or supported by the server. In particular embodiments, transportation management system 1360 may include one or more data stores. The data stores may be used to store various types of information, such as ride information, ride requestor information, ride provider information, historical information, third-party information, or any other suitable type of information. In particular embodiments, the information stored in the data stores may be organized according to specific data structures. In particular embodiments, each data store may be a relational, columnar, correlation, or any other suitable database system. Although this disclosure describes or illustrates particular types of databases, this disclosure contemplates any suitable types of databases. Particular embodiments may provide interfaces that enable a user device 1330 (which may belong to a ride requestor or provider), a transportation management system 1360, vehicle system 1340, or a third-party system 1370 to process, transform, manage, retrieve, modify, add, or delete the information stored in the data store.

In particular embodiments, transportation management system 1360 may include an authorization server (or other suitable component(s)) that allows users 1301 to opt-in to or opt-out of having their information and actions logged, recorded, or sensed by transportation management system 1360 or shared with other systems (e.g., third-party systems 1370). In particular embodiments, a user 1301 may opt-in or opt-out by setting appropriate privacy settings. A privacy setting of a user may determine what information associated with the user may be logged, how information associated with the user may be logged, when information associated with the user may be logged, who may log information associated with the user, whom information associated with the user may be shared with, and for what purposes information associated with the user may be logged or shared. Authorization servers may be used to enforce one or more privacy settings of the users 1301 of transportation management system 1360 through blocking, data hashing, anonymization, or other suitable techniques as appropriate.

In particular embodiments, third-party system 1370 may be a network-addressable computing system that may provide HD maps or host GPS maps, customer reviews, music or content, weather information, or any other suitable type of information. Third-party system 1370 may generate, store, receive, and send relevant data, such as, for example, map data, customer review data from a customer review website, weather data, or any other suitable type of data.

Third-party system 1370 may be accessed by the other computing entities of the network environment either directly or via network 1310. For example, user device 1330 may access the third-party system 1370 via network 1310, or via transportation management system 1360. In the latter case, if credentials are required to access the third-party system 1370, the user 1301 may provide such information to the transportation management system 1360, which may serve as a proxy for accessing content from the third-party system 1370.

In particular embodiments, user device 1330 may be a mobile computing device such as a smartphone, tablet computer, or laptop computer. User device 1330 may include one or more processors (e.g., CPU and/or GPU), memory, and storage. An operating system and applications may be installed on the user device 1330, such as, e.g., a transportation application associated with the transportation management system 1360, applications associated with third-party systems 1370, and applications associated with the operating system. User device 1330 may include functionality for determining its location, direction, or orientation, based on integrated sensors such as GPS, compass, gyroscope, or accelerometer. User device 1330 may also include wireless transceivers for wireless communication and may support wireless communication protocols such as Bluetooth, near-field communication (NFC), infrared (IR) communication, WI-FI, and/or 2G/3G/4G/LTE mobile communication standard. User device 1330 may also include one or more cameras, scanners, touch screens, microphones, speakers, and any other suitable input-output devices.

In particular embodiments, the vehicle 1340 may be an autonomous vehicle and equipped with an array of sensors 1344, a navigation system 1346, and a ride-service computing device 1348. In particular embodiments, a fleet of autonomous vehicles 1340 may be managed by the transportation management system 1360. The fleet of autonomous vehicles 1340, in whole or in part, may be owned by the entity associated with the transportation management system 1360, or they may be owned by a third-party entity relative to the transportation management system 1360. In either case, the transportation management system 1360 may control the operations of the autonomous vehicles 1340, including, e.g., dispatching select vehicles 1340 to fulfil ride requests, instructing the vehicles 1340 to perform select operations (e.g., head to a service center or charging/fueling station, pull over, stop immediately, self-diagnose, lock/unlock compartments, change music station, change temperature, and any other suitable operations), and instructing the vehicles 1340 to enter select operation modes (e.g., operate normally, drive at a reduced speed, drive under the command of human operators, and any other suitable operational modes).

In particular embodiments, the autonomous vehicles 1340 may receive data from and transmit data to the transportation management system 1360 and the third-party system 1370. Example of received data may include, e.g., instructions, new software or software updates, maps, 3D models, trained or untrained machine-learning models, location information (e.g., location of the ride requestor, the autonomous vehicle 1340 itself, other autonomous vehicles 1340, and target destinations such as service centers), navigation information, traffic information, weather information, entertainment content (e.g., music, video, and news) ride requestor information, ride information, and any other suitable information. Examples of data transmitted from the autonomous vehicle 1340 may include, e.g., telemetry and sensor data, determinations/decisions based on such data, vehicle condition or state (e.g., battery/fuel level, tire and brake conditions, sensor condition, speed, odometer, etc.), location, navigation data, passenger inputs (e.g., through a user interface in the vehicle 1340, passengers may send/receive data to the transportation management system 1360 and/or third-party system 1370), and any other suitable data.

In particular embodiments, autonomous vehicles 1340 may also communicate with each other as well as other traditional human-driven vehicles, including those managed and not managed by the transportation management system 1360. For example, one vehicle 1340 may communicate with another vehicle data regarding their respective location, condition, status, sensor reading, and any other suitable information. In particular embodiments, vehicle-to-vehicle communication may take place over a direct short-range wireless connection (e.g., WI-FI, Bluetooth, NFC) and/or over a network (e.g., the Internet or via the transportation management system 1360 or third-party system 1370).

In particular embodiments, an autonomous vehicle 1340 may obtain and process sensor/telemetry data. Such data may be captured by any suitable sensors. For example, the vehicle 1340 may have a Light Detection and Ranging (LiDAR) sensor array of multiple LiDAR transceivers that are configured to rotate 360°, emitting pulsed laser light and measuring the reflected light from objects surrounding vehicle 1340. In particular embodiments, LiDAR transmitting signals may be steered by use of a gated light valve, which may be a MEMs device that directs a light beam using the principle of light diffraction. Such a device may not use a gimbaled mirror to steer light beams in 360° around the autonomous vehicle. Rather, the gated light valve may direct the light beam into one of several optical fibers, which may be arranged such that the light beam may be directed to many discrete positions around the autonomous vehicle. Thus, data may be captured in 360° around the autonomous vehicle, but no rotating parts may be necessary. A LIDAR is an effective sensor for measuring distances to targets, and as such may be used to generate a three-dimensional (3D) model of the external environment of the autonomous vehicle 1340. As an example and not by way of limitation, the 3D model may represent the external environment including objects such as other cars, curbs, debris, objects, and pedestrians up to a maximum range of the sensor arrangement (e.g., 50, 130, or 200 meters). As another example, the autonomous vehicle 1340 may have optical cameras pointing in different directions. The cameras may be used for, e.g., recognizing roads, lane markings, street signs, traffic lights, police, other vehicles, and any other visible objects of interest. To enable the vehicle 1340 to “see” at night, infrared cameras may be installed. In particular embodiments, the vehicle may be equipped with stereo vision for, e.g., spotting hazards such as pedestrians or tree branches on the road. As another example, the vehicle 1340 may have radars for, e.g., detecting other vehicles and/or hazards afar. Furthermore, the vehicle 1340 may have ultrasound equipment for, e.g., parking and obstacle detection. In addition to sensors enabling the vehicle 1340 to detect, measure, and understand the external world around it, the vehicle 1340 may further be equipped with sensors for detecting and self-diagnosing the its own state and condition. For example, the vehicle 1340 may have wheel sensors for, e.g., measuring velocity; global positioning system (GPS) for, e.g., determining the vehicle's current geolocation; and/or inertial measurement units, accelerometers, gyroscopes, and/or odometer systems for movement or motion detection. While the description of these sensors provides particular examples of utility, one of ordinary skill in the art would appreciate that the utilities of the sensors are not limited to those examples. Further, while an example of a utility may be described with respect to a particular type of sensor, it should be appreciated that the utility may be achieving using any combination of sensors. For example, an autonomous vehicle 1340 may build a 3D model of its surrounding based on data from its LiDAR, radar, sonar, and cameras, along with a pre-generated map obtained from the transportation management system 1360 or the third-party system 1370. Although sensors 1344 appear in a particular location on autonomous vehicle 1340 in FIG. 13, sensors 1344 may be located in any suitable location in or on autonomous vehicle 1340. Example locations for sensors include the front and rear bumpers, the doors, the front windshield, on the side paneling, or any other suitable location.

In particular embodiments, the autonomous vehicle 1340 may be equipped with a processing unit (e.g., one or more CPUs and GPUs), memory, and storage. The vehicle 1340 may thus be equipped to perform a variety of computational and processing tasks, including processing the sensor data, extracting useful information, and operating accordingly. For example, based on images captured by its cameras and a machine-vision model, the vehicle 1340 may identify particular types of objects captured by the images, such as pedestrians, other vehicles, lanes, curbs, and any other objects of interest.

In particular embodiments, the autonomous vehicle 1340 may have a navigation system 1346 responsible for safely navigating the autonomous vehicle 1340. In particular embodiments, the navigation system 1346 may take as input any type of sensor data from, e.g., a Global Positioning System (GPS) module, inertial measurement unit (IMU), LiDAR sensors, optical cameras, radio frequency (RF) transceivers, or any other suitable telemetry or sensory mechanisms. The navigation system 1346 may also utilize, e.g., map data, traffic data, accident reports, weather reports, instructions, target destinations, and any other suitable information to determine navigation routes and particular driving operations (e.g., slowing down, speeding up, stopping, swerving, etc.). In particular embodiments, the navigation system 1346 may use its determinations to control the vehicle 1340 to operate in prescribed manners and to guide the autonomous vehicle 1340 to its destinations without colliding into other objects. Although the physical embodiment of the navigation system 1346 (e.g., the processing unit) appears in a particular location on autonomous vehicle 1340 in FIG. 13, navigation system 1346 may be located in any suitable location in or on autonomous vehicle 1340. Example locations for navigation system 1346 include inside the cabin or passenger compartment of autonomous vehicle 1340, near the engine/battery, near the front seats, rear seats, or in any other suitable location.

In particular embodiments, the autonomous vehicle 1340 may be equipped with a ride-service computing device 1348, which may be a tablet or other suitable device installed by transportation management system 1360 to allow the user to interact with the autonomous vehicle 1340, transportation management system 1360, other users 1301, or third-party systems 1370. In particular embodiments, installation of ride-service computing device 1348 may be accomplished by placing the ride-service computing device 1348 inside autonomous vehicle 1340 and configuring it to communicate with the vehicle 1340 via a wire or wireless connection (e.g., via Bluetooth). Although FIG. 13 illustrates a single ride-service computing device 1348 at a particular location in autonomous vehicle 1340, autonomous vehicle 1340 may include several ride-service computing devices 1348 in several different locations within the vehicle. As an example and not by way of limitation, autonomous vehicle 1340 may include four ride-service computing devices 1348 located in the following places: one in front of the front-left passenger seat (e.g., driver's seat in traditional U.S. automobiles), one in front of the front-right passenger seat, one in front of each of the rear-left and rear-right passenger seats. In particular embodiments, ride-service computing device 1348 may be detachable from any component of autonomous vehicle 1340. This may allow users to handle ride-service computing device 1348 in a manner consistent with other tablet computing devices. As an example and not by way of limitation, a user may move ride-service computing device 1348 to any location in the cabin or passenger compartment of autonomous vehicle 1340, may hold ride-service computing device 1348 in the user's lap, or handle ride-service computing device 1348 in any other suitable manner. Although this disclosure describes providing a particular computing device in a particular manner, this disclosure contemplates providing any suitable computing device in any suitable manner.

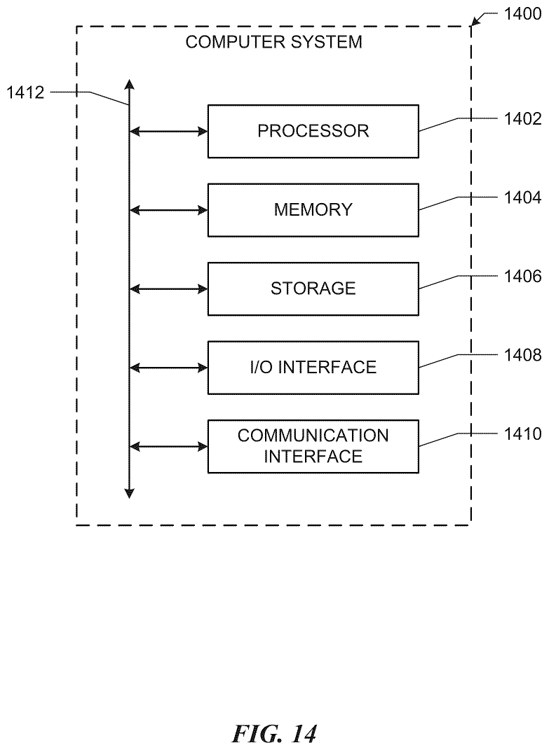

FIG. 14 illustrates an example computer system 1400. In particular embodiments, one or more computer systems 1400 perform one or more steps of one or more methods described or illustrated herein. In particular embodiments, one or more computer systems 1400 provide functionality described or illustrated herein. In particular embodiments, software running on one or more computer systems 1400 performs one or more steps of one or more methods described or illustrated herein or provides the functionality described or illustrated herein. Particular embodiments include one or more portions of one or more computer systems 1400. Herein, references to a computer system may encompass a computing device, and vice versa, where appropriate. Moreover, references (to a computer system may encompass one or more computer systems, where appropriate.

This disclosure contemplates any suitable number of computer systems 1400. This disclosure contemplates computer system 1400 taking any suitable physical form. As example and not by way of limitation, computer system 1400 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 1400 may include one or more computer systems 1400; be unitary or distributed; span multiple locations; span multiple machines; span multiple data centers; or reside in a cloud, which may include one or more cloud components in one or more networks. Where appropriate, one or more computer systems 1400 may perform without substantial spatial or temporal limitation one or more steps of one or more methods described or illustrated herein. As an example and not by way of limitation, one or more computer systems 1400 may perform in real time or in batch mode one or more steps of one or more methods described or illustrated herein. One or more computer systems 1400 may perform at different times or at different locations one or more steps of one or more methods described or illustrated herein, where appropriate.

In particular embodiments, computer system 1400 includes a processor 1402, memory 1404, storage 1406, an input/output (I/O) interface 1408, a communication interface 1410, and a bus 1412. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1402 includes hardware for executing instructions, such as those making up a computer program. As an example and not by way of limitation, to execute instructions, processor 1402 may retrieve (or fetch) the instructions from an internal register, an internal cache, memory 1404, or storage 1406; decode and execute them; and then write one or more results to an internal register, an internal cache, memory 1404, or storage 1406. In particular embodiments, processor 1402 may include one or more internal caches for data, instructions, or addresses. This disclosure contemplates processor 1402 including any suitable number of any suitable internal caches, where appropriate. As an example and not by way of limitation, processor 1402 may include one or more instruction caches, one or more data caches, and one or more translation lookaside buffers (TLBs). Instructions in the instruction caches may be copies of instructions in memory 1404 or storage 1406, and the instruction caches may speed up retrieval of those instructions by processor 1402. Data in the data caches may be copies of data in memory 1404 or storage 1406 for instructions executing at processor 1402 to operate on; the results of previous instructions executed at processor 1402 for access by subsequent instructions executing at processor 1402 or for writing to memory 1404 or storage 1406; or other suitable data. The data caches may speed up read or write operations by processor 1402. The TLBs may speed up virtual-address translation for processor 1402. In particular embodiments, processor 1402 may include one or more internal registers for data, instructions, or addresses. This disclosure contemplates processor 1402 including any suitable number of any suitable internal registers, where appropriate. Where appropriate, processor 1402 may include one or more arithmetic logic units (ALUs); be a multi-core processor; or include one or more processors 1402. Although this disclosure describes and illustrates a particular processor, this disclosure contemplates any suitable processor.

In particular embodiments, memory 1404 includes main memory for storing instructions for processor 1402 to execute or data for processor 1402 to operate on. As an example and not by way of limitation, computer system 1400 may load instructions from storage 1406 or another source (such as another computer system 1400) to memory 1404. Processor 1402 may then load the instructions from memory 1404 to an internal register or internal cache. To execute the instructions, processor 1402 may retrieve the instructions from the internal register or internal cache and decode them. During or after execution of the instructions, processor 1402 may write one or more results (which may be intermediate or final results) to the internal register or internal cache. Processor 1402 may then write one or more of those results to memory 1404. In particular embodiments, processor 1402 executes only instructions in one or more internal registers or internal caches or in memory 1404 (as opposed to storage 1406 or elsewhere) and operates only on data in one or more internal registers or internal caches or in memory 1404 (as opposed to storage 1406 or elsewhere). One or more memory buses (which may each include an address bus and a data bus) may couple processor 1402 to memory 1404. Bus 1412 may include one or more memory buses, as described in further detail below. In particular embodiments, one or more memory management units (MMUs) reside between processor 1402 and memory 1404 and facilitate accesses to memory 1404 requested by processor 1402. In particular embodiments, memory 1404 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1404 may include one or more memories 1404, where appropriate. Although this disclosure describes and illustrates particular memory, this disclosure contemplates any suitable memory.

In particular embodiments, storage 1406 includes mass storage for data or instructions. As an example and not by way of limitation, storage 1406 may include a hard disk drive (HDD), a floppy disk drive, flash memory, an optical disc, a magneto-optical disc, magnetic tape, or a Universal Serial Bus (USB) drive or a combination of two or more of these. Storage 1406 may include removable or non-removable (or fixed) media, where appropriate. Storage 1406 may be internal or external to computer system 1400, where appropriate. In particular embodiments, storage 1406 is non-volatile, solid-state memory. In particular embodiments, storage 1406 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1406 taking any suitable physical form. Storage 1406 may include one or more storage control units facilitating communication between processor 1402 and storage 1406, where appropriate. Where appropriate, storage 1406 may include one or more storages 1406. Although this disclosure describes and illustrates particular storage, this disclosure contemplates any suitable storage.

In particular embodiments, I/O interface 1408 includes hardware, software, or both, providing one or more interfaces for communication between computer system 1400 and one or more I/O devices. Computer system 1400 may include one or more of these I/O devices, where appropriate. One or more of these I/O devices may enable communication between a person and computer system 1400. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1408 for them. Where appropriate, I/O interface 1408 may include one or more device or software drivers enabling processor 1402 to drive one or more of these I/O devices. I/O interface 1408 may include one or more I/O interfaces 1408, where appropriate. Although this disclosure describes and illustrates a particular I/O interface, this disclosure contemplates any suitable I/O interface.

In particular embodiments, communication interface 1410 includes hardware, software, or both providing one or more interfaces for communication (such as, for example, packet-based communication) between computer system 1400 and one or more other computer systems 1400 or one or more networks. As an example and not by way of limitation, communication interface 1410 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1410 for it. As an example and not by way of limitation, computer system 1400 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1400 may communicate with a wireless PAN (WPAN) (such as, for example, a Bluetooth WPAN), a WI-FI network, a WI-MAX network, a cellular telephone network (such as, for example, a Global System for Mobile Communications (GSM) network), or other suitable wireless network or a combination of two or more of these. Computer system 1400 may include any suitable communication interface 1410 for any of these networks, where appropriate. Communication interface 1410 may include one or more communication interfaces 1410, where appropriate. Although this disclosure describes and illustrates a particular communication interface, this disclosure contemplates any suitable communication interface.

In particular embodiments, bus 1412 includes hardware, software, or both coupling components of computer system 1400 to each other. As an example and not by way of limitation, bus 1412 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1412 may include one or more buses 1412, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

1. A method comprising, by a computing system associated with a transportation management system:
receiving, by the computing system, a ride request from a ride requestor;
accessing, by the computing system, a user preference profile of the ride requestor, the user preference profile indicating at least one ride preference of the ride requestor;
matching, by the computing system, the ride requestor to a ride provider associated with the transportation management system based on the at least one ride preference of the ride requestor;
sending, by the computing system, the at least one ride preference to a computing device of a vehicle associated with the ride provider;
determining, by the computing system, a current location of each of the ride requestor and the ride provider; and
in response to determining that the current location of each of the ride requestor and the ride provider is within a threshold distance, instructing, by the computing system, the computing device of the vehicle to adjust one or more settings of one or more in-vehicle devices according to the at least one ride preference of the ride requestor.

2. The method of claim 1, wherein the threshold distance varies based on a type of adjustment to be performed.

3. The method of claim 1, wherein adjusting the one or more settings of the one or more in-vehicle devices comprises:
changing media content played inside the vehicle according to the at least one ride preference of the ride requestor.

4. The method of claim 3, wherein the media content comprises one or more of an audio content, a video content, or a news content.

5. The method of claim 1, wherein adjusting the one or more settings of the one or more in-vehicle devices comprises:
changing a temperature inside the vehicle according to the at least one ride preference of the ride requestor.

6. The method of claim 1, wherein adjusting the one or more settings of the one or more in-vehicle devices comprises:
opening or closing one or more windows of the vehicle according to the at least one ride preference of the ride requestor.

7. The method of claim 1, wherein adjusting the one or more settings of the one or more in-vehicle devices comprises:
reconfiguring one or more seats of the vehicle according to the at least one ride preference of the ride requestor.

8. The method of claim 1, further comprising:
determining whether the vehicle has reached a destination associated with the ride request; and
in response to determining that the vehicle has reached the destination and the ride requestor has exited the vehicle, instructing the computing device of the vehicle to reset the one or more settings of the one or more in-vehicle devices.

9. The method of claim 8, wherein resetting the one or more settings of the one or more in-vehicle devices comprises:
switching the one or more settings from a current state to a previous state, wherein:
the current state corresponds to first settings of the one or more in-vehicle devices according to the at least one ride preference of the ride requestor, and
the previous state corresponds to second settings of the one or more in-vehicle devices according to the ride provider.

10. The method of claim 1, wherein matching the ride requestor to the ride provider comprises:
determining a plurality of ride providers;
determining a number of ride preferences of the ride requestor offered by each of the plurality of ride providers;
ranking the plurality of ride providers based on the number of ride preferences of the ride requestor offered by each of the plurality of ride providers; and
selecting the ride provider based on the ranking.

11. The method of claim 1, wherein the at least one ride preference comprises one of an audio preference, a video preference, a volume preference, a temperature preference, a conversation preference, and a language preference.

12. The method of claim 1, wherein the at least one ride preference of the ride requestor is predicted using a machine-learning model.

13. The method of claim 12, wherein the machine-learning model uses information associated with the ride requestor and contextual information relating to the ride request to predict the at least one ride preference of the ride requestor.

14. The method of claim 13, wherein the contextual information relating to the ride request comprises one or more of current weather condition or temperature, time of day, traffic condition, or actions of the ride requestor with respect to making the ride request.

15. A computing system comprising:
one or more processors; and
one or more computer-readable non-transitory storage media coupled to the one or more processors, the one or more computer-readable non-transitory storage media comprising instructions that, when executed by the one or more processors, cause the computing system to perform operations comprising:
receiving a ride request from a ride requestor;
accessing a user preference profile of the ride requestor, the user preference profile indicating at least one ride preference of the ride requestor;
matching the ride requestor to a ride provider associated with a transportation management system based on the at least one ride preference of the ride requestor;
sending the at least one ride preference to a computing device of a vehicle associated with the ride provider;
determining a current location of each of the ride requestor and the ride provider; and
in response to determining that the current location of each of the ride requestor and the ride provider is within a threshold distance, instructing the computing device of the vehicle to adjust one or more settings of one or more in-vehicle devices according to the at least one ride preference of the ride requestor.

16. The computing system of claim 15, wherein adjusting the one or more settings of the one or more in-vehicle devices comprises:
changing media content played inside the vehicle according to the at least one ride preference of the ride requestor.

17. The computing system of claim 15, wherein the instructions further comprise instructions to cause the computing system to perform operations comprising:
determining whether the vehicle has reached a destination associated with the ride request; and
in response to determining that the vehicle has reached the destination and the ride requestor has exited the vehicle, instructing the computing device of the vehicle to reset the one or more settings of the one or more in-vehicle devices.

18. One or more computer-readable non-transitory storage media including instructions that, when executed by one or more processors, cause the one or more processors to perform operations comprising:
receiving a ride request from a ride requestor;
accessing a user preference profile of the ride requestor, the user preference profile indicating at least one ride preference of the ride requestor;
matching the ride requestor to a ride provider associated with a transportation management system based on the at least one ride preference of the ride requestor;
sending the at least one ride preference to a computing device of a vehicle associated with the ride provider;
determining a current location of each of the ride requestor and the ride provider; and
in response to determining that the current location of each of the ride requestor and the ride provider is within a threshold distance, instructing the computing device of the vehicle to adjust one or more settings of one or more in-vehicle devices according to the at least one ride preference of the ride requestor.

19. The one or more computer-readable non-transitory storage media of claim 18, wherein adjusting the one or more settings of the one or more in-vehicle devices comprises:
changing media content played inside the vehicle according to the at least one ride preference of the ride requestor.

20. The one or more computer-readable non-transitory storage media of claim 18, wherein the instructions further comprise instructions to cause the one or more processors to perform operations comprising:
determining whether the vehicle has reached a destination associated with the ride request; and
in response to determining that the vehicle has reached the destination and the ride requestor has exited the vehicle, instructing the computing device of the vehicle to reset the one or more settings of the one or more in-vehicle devices.
